(19) United States
(12) Patent Application Publication
(10) Pub. No.: US 2008/0269275 A1
(43) Pub. Date: Oct. 30, 2008

(54) Novel Mchr1 Antagonists and Their Use for the Treatment of Mchr1 Mediated Conditions and Disorders

(75) Inventors: Dean Brown, Wilmington, DE (US); William Blackwell, Wilmington, DE (US); Frances M. McLaren, Wilmington, DE (US); Volker Schnecke, Molndal (SE); Reed W. Smith, Wilmington, DE (US); Gary Steelman, Wilmington, DE (US); Xia Wang, Wilmington, DE (US); Rebecca Urbanek, Wilmington, DE (US); Steven Wesolowski, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/914,400

(22) PCT Filed: May 29, 2006
(86) PCT No.: PCT/SE06/00621

§ 371 Date: Nov. 14, 2007

Related U.S. Application Data
(60) Provisional application No. 60/685,832, filed on May 31, 2005.

(51) Int. Cl. A61K 31/439; C07D 471/08; C07D 211/06; A61K 31/445

(52) U.S. Cl. 514/294; 546/93; 546/216; 514/327

(57) ABSTRACT

Compounds of Formula I

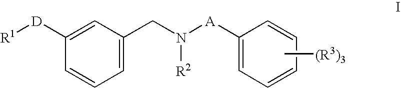

wherein R¹, D, R², A and R³ are as described in the specification, pharmaceutically-acceptable salts, methods of making, pharmaceutical compositions containing and methods for using the same.

### FIELD OF THE INVENTION

The present invention relates to compounds, compositions and methods useful in the treatment or prevention of conditions or disorders related to mood changes, anxiety, depression, obesity and related disorders, eating disorders, psychiatric disorders, neurological disorders and pain.

### BACKGROUND OF THE INVENTION

Melanin-concentrating hormone (MCH) is a cyclic neuropeptide involved in the regulation of several functions in the brain. It has been found to be a major regulator of eating behavior and energy homeostasis and is the natural ligand for the 353-amino acid orphan G-protein-coupled-receptor (GPCR) termed SLC-1 (also known as GPR24). SLC-1 is sequentially homologous to the somatostatin receptors, is frequently referred to as “melanin-concentrating hormone receptor” (MCH receptor type 1, MCH1 receptor, or MCHR1), Chambers et al., Nature 400:261-65 (1999); Saito et al., Nature 400:265-69 (1999); and Saito et al., TEM 11(8):299-303 (2000).

In mice lacking the MCH1 receptor, there is no increased feeding response to MCH, and a lean phenotype is seen, suggesting that this receptor is responsible for mediating the feeding effect of MCH, Marsh et al., Proc Natl Acad Sci USA. 99(5):3240-5, (2002). MCH receptor antagonists have also been shown to block the feeding effects of MCH (Takekawa et al, Eur. J Pharmacol. 438(3):129-35, (2002), and to reduce body weight & adiposity in diet-induced obese rats (Borowsky et al., Nat Med. 8(8):825-30, (2002). The conservation of distribution and sequence of MCH1 receptors suggest a similar role for this receptor in man and rodent species. Hence, MCH receptor antagonists have been proposed as a treatment for obesity and other disorders characterized by excessive eating and body weight.

Emerging evidence also suggests that MCHR1 plays a role in the regulation of mood and stress. Within the central nervous system, MCHR1 mRNA and protein are distributed in various hypothalamic nuclei including the paraventricular nucleus (PVN), the nucleus accumbens shell, and several limbic structures including hippocampus, septum, amygdala, locus coeruleus and dorsal raphe nucleus, all of which are thought to be involved in the regulation of emotion and stress, Hervieu et al., European Journal of Neuroscience. 12(4):1194-216, (2000); Saito et al., Journal of Comparative Neurology. 435(1):26-40, (2001); Borowsky et al.

Introduction of MCH into the medial preoptic area has been reported to induce anxiety, Gonzalez et al., Peptides. 1996;17(1):171-7, (1996), although contrary anxiolytic-like effects of MCH injection have also been reported, Kela et al., Regulatory Peptides. 114(2-3):109-14, (2003). Injection of MCH into the nucleus accumbens shell, in which MCHR1 is abundant, decreased mobility in a forced swim test in rats, suggesting a depressive effect, Sears et al., J Neurosci. 25(11):2933-40 (2005). Also, it has been reported that MCHR1 antagonists exhibited antidepressant and anxiolytic-like effects in rodents, suggesting a role for MCHR1 in depression and anxiety, Borowsky et al.; Chaki et al., JPET 313:831-839, (2005).

### DESCRIPTION OF THE INVENTION

The present invention provides compounds and compositions, and methods of use thereof to treat or prevent conditions and disorders mediated by MCHR1. Such compounds are antagonists of MCHR1 and have structures in accord with Formula I:

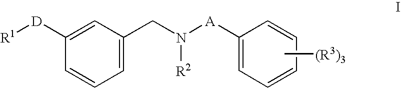

wherein:

D is selected from —CH₂— or —O—, and

R¹ is selected from —C₁₋₆alkylene-NR⁵R⁶ wherein R⁵ and R⁶ are independently at each occurrence selected from hydrogen or -C₁₋₆alkyl, or R⁵ and R⁶ together with the N to which they are attached are selected from morpholino or a moiety of Formula II

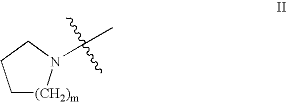

where m is 1, 2 or 3, and the moiety of Formula II may be substituted with ═O; or, R¹ is selected from:

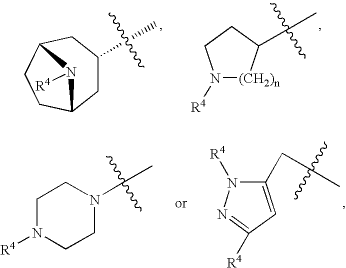

wherein R⁴ is selected from hydrogen, -C₁₋₆alkyl, -C₃₋₈cycloalkyl, -C₃₋₈cyclooxyalkyl or benzyl and n is 1, 2 or 3,

R² is selected from hydrogen, -C₁₋₆alkyl or C₃₋₈cycloalkyl;

A is selected from —CH₂— or —C(═O)—;

R³ is selected independently at each occurrence from hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, -C₁₋₆alkyl, -C₂₋₆alkenyl, -C₂₋₆alkynyl, -C₁₋₆alkoxy, -C₃₋₈cycloalkyl, —O—CH₂—O—, or —G—Ar,

wherein G is —O—, —CH₂—, —O—CH₂— or a bond, and

Ar is selected from a 5- or 6-membered aromatic or heteroaromatic ring having 0, 1 or 2 nitrogen atoms, 0 or 1 oxygen atoms, and 0 or 1 sulfur atoms, or is selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein Ar is unsubstituted or has 1, 2 or 3 substituents independently selected at each occurrence from -C₁₋₆alkyl, -C₂₋₆alkenyl, -C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷ or —CO₂R⁷;

wherein R⁷ and R⁸ are independently selected from hydrogen, -C₁₋₆alkyl, -C₁₋₆alkoxy or -C₃₋₈cycloalkyl.

The invention also encompasses stereoisomers, enantiomers, in vivo-hydrolysable precursors and pharmaceutically-acceptable salts of compounds of Formula I, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes. In particular, the present invention provides compounds, compositions containing them, and methods using them for treating or preventing conditions and disorders associated with mood changes, anxiety, depression, obesity and related disorders, eating disorders, psychiatric disorders, neurological disorders and pain.

Compounds of the invention are those in accord with Formula I:

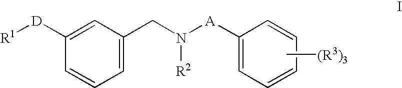

wherein:

D is selected from —CH₂— or —O—, and

R¹ is selected from -C₁₋₆alkylene-NR⁵R⁶ wherein R⁵ and R⁶ are independently at each occurrence selected from hydrogen or -C₁₋₆alkyl, or R⁵ and R⁶ together with the N to which they are attached are selected from morpholino or a moiety of Formula II

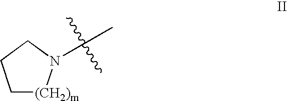

where m is 1, 2 or 3, and the moiety of Formula II may be substituted with ═O; or, R¹ is selected from:

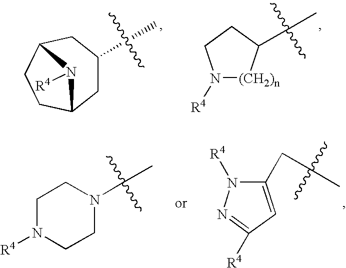

wherein R⁴ is selected from hydrogen, -C₁₋₆alkyl, -C₃₋₈cycloalkyl, -C₃₋₈cyclooxyalkyl or benzyl and n is 1, 2 or 3,

R² is selected from hydrogen, -C₁₋₆alkyl or C₃₋₈cycloalkyl;

A is selected from —CH₂— or —C(═O)—;

R³ is selected independently at each occurrence from hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, -C₁₋₆alkyl, -C₂₋₆alkenyl, -C₂₋₆alkynyl, -C₁₋₆alkoxy, -C₃₋₈cycloalkyl, —O—CH₂—O—, or —G—Ar,

wherein G is —O—, —CH₂—, —O—CH₂— or a bond, and

Ar is selected from a 5- or 6-membered aromatic or heteroaromatic ring having 0, 1 or 2 nitrogen atoms, 0 or 1 oxygen atoms, and 0 or 1 sulfur atoms, or is selected from an 8-, 9- or 10-membered fused aromatic or heteroaromatic ring system having 0, 1, 2 or 3 nitrogen atoms, 0 or 1 oxygen atoms, and 0 or 1 sulfur atoms;

wherein Ar is unsubstituted or has 1, 2 or 3 substituents independently selected at each occurrence from -C₁₋₆alkyl, -C₂₋₆alkenyl, -C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷ or —CO₂R⁷;

wherein R⁷ and R⁸ are independently selected from hydrogen, -C₁₋₆alkyl, -C₁₋₆alkoxy or -C₃₋₈cycloalkyl,

or in vivo-hydrolysable precursors or pharmaceutically-acceptable salts thereof, with the proviso that said compound is not N-[3-(1-methyl-piperidin-4-yloxy)-benzyl]-3-phenoxy-benzamide or N-[3-(1-methyl-piperidin-4-yloxy)-benzyl]-4-phenoxy-benzamide.

Particular compounds of the invention are those in accord with Formula I:

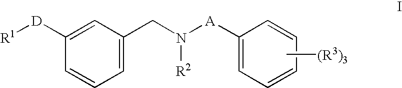

wherein:

where D is —O—, and

R¹, R² and R³ are as heretofore defined.

Other particular compounds of the invention are those in accord with Formula I:

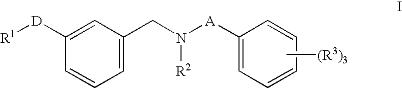

wherein:

A is —C(═O)— and D, R¹, R² and R³ are as heretofore defined.

Yet other particular compounds of the invention are those in accord with Formula I:

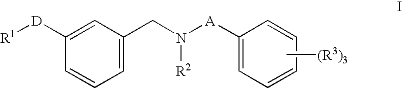

wherein:

where D is selected from —CH₂— or —O—, and

R¹ is selected from:

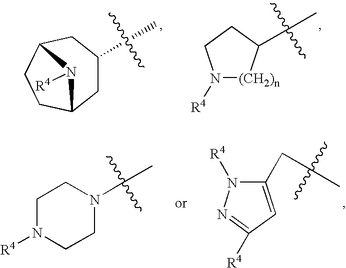

wherein R², A, R³ and R⁴ are as heretofore defined.

Exemplary compounds of the invention are described herein.

In a further aspect the invention relates to compounds described herein wherein one or more of the atoms is a radioisotope of the same element. In a particular form of this aspect of the invention the compound is labeled with tritium. Such radio-labeled compounds are synthesized either by incorporating radio-labeled starting materials or, in the case of tritium, exchange of hydrogen for tritium by known methods. Known methods include (1) electrophilic halogenation, followed by reduction of the halogen in the presence of a tritium source, for example, by hydrogenation with tritium gas in the presence of a palladium catalyst, or (2) exchange of hydrogen for tritium performed in the presence of tritium gas and a suitable organometallic (e.g. palladium) catalyst.

Compounds of the invention labeled with tritium are useful for the discovery of novel medicinal compounds which bind to and modulate the activity, by agonism, partial agonism, or antagonism, of an MCH1 receptor. Such tritium-labeled compounds may be used in assays that measure the displacement of such compounds to assess the binding of ligands that bind to MCH1 receptors.

In a further aspect the invention relates to compounds described herein additionally comprising one or more atoms of a radioisotope. In a particular form of this aspect of the invention the compound comprises a radioactive halogen. Such radio-labeled compounds are synthesized by incorporating radio-labeled starting materials by known methods. Particular embodiments of this aspect of the invention are those in which the radioisotope is selected from ¹⁸F, ¹²³I, ¹²⁵I, ¹³¹I, ⁷⁵Br, ⁷⁶Br, ⁷⁷Br or ⁸²Br. A most particular embodiment of this aspect of the invention is that in which the radioisotope is ¹⁸F.

In another aspect the invention relates to compounds in accord with Formula I described herein including N-[3-(1-methyl-piperidin-4-yloxy)-benzyl]-3-phenoxy-benzamide and N-[3-(1-methyl-piperidin-4-yloxy)-benzyl]-4-phenoxy-benzamide and the use of such compounds in therapy and in compositions useful for therapy.

In another aspect the invention encompasses the use of antagonist compounds described herein for the therapy of diseases mediated through the action of MCH1 receptors. A more particular aspect of the invention relates to the use of the compounds for the therapy of diseases mediated through the action of MCH1 receptors.

Another aspect of the invention encompasses a method of treatment or prophylaxis of diseases or conditions in which modulation of the MCH1 receptor is beneficial which method comprises administering a therapeutically-effective amount of an antagonistic compound of the invention to a subject suffering from said disease or condition.

One embodiment of this aspect of the invention is a method of treatment or prophylaxis, wherein the disorder is a mood disorder, anxiety, or depression. More particular embodiments encompass treatment or prophylaxis of anxiety, generalized anxiety disorder, panic attacks, panic disorder, obsessive-compulsive disorder, depression and bipolar disorders. Another embodiment of this aspect of the invention provides compounds, which are useful in treating obesity and related disorders, eating disorders, psychiatric disorders, neurological disorders and pain.

According to another aspect of the invention, a method is provided of treating obesity, psychiatric disorders, anxiety, anxio-depressive disorders, depression, bipolar disorder, ADHD, cognitive disorders, memory disorders, schizophrenia, epilepsy, and related conditions, and neurological disorders and pain related disorders, comprising administering a pharmacologically effective amount of a compound of Formula I to a patient in need thereof.

Still a further aspect of the invention, provides compounds useful for treating obesity, type II diabetes, metabolic syndrome and for preventing type II diabetes comprising administering a pharmacologically effective amount of a compound of Formula I to a patient in need thereof.

Yet another aspect of the invention, provides processes for the preparation of compounds of Formula I.

Compounds of the present invention have the advantage that they may be more potent, more selective, more efficacious in vivo, be less toxic, be longer acting, produce fewer side effects, be more easily absorbed, be less metabolized and/or have a better pharmacokinetic profile than, or have other useful pharmacological or physicochemical properties over known compounds.

Another embodiment of this aspect of the invention is a pharmaceutical composition comprising a compound of the invention and a pharmaceutically-acceptable diluent, lubricant or carrier.

A further aspect of the invention relates to a pharmaceutical composition useful for treating or preventing a condition or disorder mentioned herein arising from dysfunction of MCH1 receptors in a mammal, preferably a human, comprising an amount of an antagonistic compound of the invention, an enantiomer thereof or a pharmaceutically-acceptable salt thereof, effective in treating or preventing such disorder or condition, and pharmaceutically-acceptable additives carrier.

A further aspect of the invention is the use of a compound according to the invention, an enantiomer thereof or a pharmaceutically-acceptable salt thereof, for the treatment or prophylaxis of a disease or condition in which modulation of the MCH1 receptor is beneficial. Particular diseases and conditions that may be treated are mood changes, anxiety or depression. More particular embodiments encompass uses of a compound for treatment or prophylaxis of anxiety, generalized anxiety disorder, panic attacks, panic disorder, obsessive-compulsive disorder, depression and bipolar disorders. Yet another embodiment of this aspect of the invention provides the use of compounds for treating obesity and related disorders, eating disorders, psychiatric disorders, neurological disorders and pain.

A further aspect of the invention is the use of a compound according to the invention, an enantiomer thereof or a pharmaceutically-acceptable salt thereof, in the manufacture of a medicament for the treatment or prophylaxis of the diseases or conditions mentioned herein.

A particular embodiment of this aspect of the invention is the use of a compound of the invention in the manufacture of a medicament for treatment or prophylaxis of mood disorders, anxiety, or depression. More particular embodiments encompass use of a compound in the manufacture of a medicament for the treatment or prophylaxis of anxiety, generalized anxiety disorder, panic attacks, panic disorder, obsessive-compulsive disorder, depression and bipolar disorders. Yet another embodiment of this aspect of the invention provides use of a compound in the manufacture of a medicament for the treatment of obesity and related disorders, eating disorders, psychiatric disorders, neurological disorders and pain.

For the uses, methods, medicaments and compositions mentioned herein the amount of compound used and the dosage administered will, of course, vary with the compound employed, the mode of administration and the treatment desired. However, in general, satisfactory results are obtained when the compounds of the invention are administered at a daily dosage of about 0.1 mg to about 20 mg/kg of animal body weight. Such doses may be given in divided doses 1 to 4 times a day or in sustained release form. For man, the total daily dose is in the range of from 5 mg to 1,400 mg, more preferably from 10 mg to 100 mg, and unit dosage forms suitable for oral administration comprise from 2 mg to 1,400 mg of the compound admixed with a solid or liquid pharmaceutical carriers, lubricants and diluents.

Compounds of the invention, enantiomers thereof, and pharmaceutically-acceptable salts thereof, may be used on their own or in the form of appropriate medicinal preparations for enteral or parenteral administration. According to a further aspect of the invention, there is provided a pharmaceutical composition including preferably less than 80% and more preferably less than 50% by weight of a compound of the invention in admixture with an inert pharmaceutically-acceptable diluent, lubricant or carrier.

Examples of diluents, lubricants and carriers are:
for tablets and dragees: lactose, starch, talc, stearic acid;
for capsules: tartaric acid or lactose;
for injectable solutions: water, alcohols, glycerin, vegetable oils;
for suppositories: natural or hardened oils or waxes.

There is also provided a process for the preparation of such a pharmaceutical composition which process comprises mixing or compounding the ingredients together and forming the mixed ingredients into tablets or suppositories, encapsulating the ingredients in capsules or dissolving the ingredients to form injectable solutions.

Some compounds of the invention may exist in tautomeric, enantiomeric, stereoisomeric or geometric isomeric forms, all of which are included within the scope of the invention. The various optical isomers may be isolated by separation of a racemic mixture of the compounds using conventional techniques, e.g. fractional crystallization, or chiral HPLC. Alternatively the individual enantiomers may be made by reaction of the appropriate optically active starting materials under reaction conditions which will not cause racemization.

Pharmaceutically-acceptable derivatives include solvates and salts. For example, the compounds of the invention can form acid addition salts with acids, such as the conventional pharmaceutically-acceptable acids, for example, maleic, hydrochloric, hydrobromic, phosphoric, acetic, fumaric, salicylic, citric, lactic, mandelic, tartaric and methanesulfonic acids.

### Assay Methods:

### MCH Binding Assay:

Binding of Melanin Concentrating Hormone (MCH) may be measured with a radioligand-binding assay employing [¹²⁵I]MCH and membranes expressing human Melanin Concentrating Hormone receptor 1 (MCHR1). Ligands that bind to MCHR1 may be identified by their ability to compete with the binding of [¹²⁵I]MCH.

[¹²⁵I]MCH may be purchased from Amersham BioSource (Cat # Im344-25 μCi). Membranes (3.8 mg/mL, cat#ES-370-M, batch 1346) may be prepared from CHOK1 cells expressing human MCH receptor 1 such as those obtainable from EuroScreen. Trizma, BSA, NaCl, and MgCl₂6H₂O were from Sigma. Human MCH was purchased from Bachem (0.5 mg, cat # H-1482).

Assays may be performed in BSA pretreated plates with 2 μg membranes per well. Saturation binding assays may be run in 50 mM Tris, pH 7.4, containing 3 mM MgCl₂ and 0.5 mg/mL BSA. To perform an assay, 20 μL of 2-fold serially diluted radioligand [¹²⁵I]MCH is added to wells of a shallow 96-well plate. This is followed by addition of 180 μL of assay buffer containing membranes at a final protein concentration of 15 μg/mL. The mixture is incubated at room temperature for 1 h before being filtered through a 96 well filter-bottom plate (GF/B), previously soaked in 0.1% BSA for at least 3 h. Collected membranes are washed 3 times with 300 μL/well of wash buffer (50 mM Tris, pH 7.4, containing 5 mM MgCl₂ and 50 mM NaCl), and then dried in air overnight or at 60° C. ¹²⁵I is measured by scintillation counting.

[¹²⁵I]MCH binding assays performed in the presence of test compounds, either at fixed or a series of concentrations, may be employed in a ligand competition binding assay. For dose-response assays, compounds may be 3-fold serially diluted in an assay plate to produce a range of concentrations. For single point assays, [¹²⁵I]MCH and membranes may be pre-mixed and then transferred to an assay plates with respective final membrane protein and radioligand concentrations of 20 μg/mL and 0.04 nM.

For analysis, cpm are converted to dpm, and nM radioligand concentration is calculated using vendor-provided specific radioactivity.

Saturation binding data may be analyzed using equation (1):

$$B = \frac{{B}_{max} [ [ {}^{125} I ] MCH ]}{{K}_{d} + \underline{[ [ {}^{125} I ] MCH ]}} \qquad ( 1 )$$

where B is concentration of bound ligand, Bₘₐₓ is the maximum concentration of bound ligand, and K_{d} is the dissociation constant for ligand.

Percent inhibition (% Inh) may be calculated using equation (2):

$$\% Inh = 100 / ( 1 - \frac{( {counts}_{sample} - {counts}_{negative} )}{( {counts}_{positive} - {counts}_{negative} )} ) \qquad ( 2 )$$

IC₅₀ values may be calculated by conventional methods using non-linear squares analysis.

For compounds of the invention, IC₅₀ values obtained by binding assays will be found to be less than 10 μM.

### MCHR1 Receptor Activation Assay:

Melanin Concentrating Hormone Receptor 1 (MCHR1) is a G-protein coupled receptor that interacts with heterotrimeric G proteins containing a Gα_{i/o} subunit. Binding of MCH to MCHR1 results in the exchange of GDP for GTP on the Gα_{i/o} proteins associated with the activated receptor. This activation can be quantified by measuring the amount of a GTP analog, GTPγ³⁵S, bound to the membrane-associated receptor. GTPγ³⁵S is not hydrolyzed by the intrinsic GTPase activity of a G-protein but instead forms a stable complex. Activation of MCH1 receptors may thus be quantified by measuring the amount of GTPγ³⁵S bound to membranes prepared from cells expressing such receptors. Membranes may be isolated by filtration or may be bound on SPA beads (Amersham). Bound GTPγ³⁵S may then be quantified by determining the amount of ³⁵S present. Inhibition of MCH binding by a competing ligand may thus be assessed by a decrease in the amount of GTPγ³⁵S bound to membranes in the presence of such a competing ligand.

For compounds of the invention, IC₅₀ values obtained with a GTPγ³⁵S assay will be found to be less than 50 μM.

### Abbreviations and Definitions

Terms and abbreviations used herein have their conventional meaning, unless otherwise defined.

The term “MCHR” refers to the melanin-concentrating hormone receptor protein 1 (MCHR1), unless otherwise stated.

The terms “treat”, “treating” and “treatment” refer to modulation of a disease and/or its attendant symptoms.

The terms “prevent”, “preventing” and “prevention” refer to decreasing or eliminating a disease and/or its attendant symptoms.

As used herein, the term “MCHR-mediated condition or disorder” and the like refers to a condition or disorder amenable to modulation by an MCHR active agent.

The term “therapeutically-effective amount” refers to that amount of a compound sufficient to modulate one or more of the symptoms of the condition or disorder being treated.

The term “anxiety disorder” refers to an emotional and/or behavioral disturbance characterized by persistent and pervasive worry or restlessness, tension or irritability for no clear reason. An anxiety disorder may be accompanied by tachycardia or dyspnea. Exemplary anxiety disorders include anxiety, generalized anxiety disorder, panic attacks, panic disorder and obsessive-compulsive disorder (OCD).

The term “mood disorder” refers to an emotional and/or behavioral disturbance characterized by persistent and pervasive bouts of euphoria and/or depression. Exemplary mood disorders include depression and bipolar disorders. Anxiety is frequently associated with mood disorders such as depression.

AcOH=Acetic acid

DMF=N,N-Dimethylformamide

DCM=Dichloromethane

DIEA=Diisopropyl ethyl amine

DMSO=Dimethylsulfoxide

EDC=N-(3-Dimethylaminopropyl)-N′-ethylcarbodiimide

EDCI=1-(3-Dimethylaminopropyl-)-3-ethylcarbodiimide hydrochloride

MeOH=Methanol

NMP=N-methyl pyrrolidine

PS-CO₃²⁻=Polystyrene bound carbonate

PS-DIBA=Polystyrene bound diisopropyl ethyl amine

PS-CNBH₄=Polystyrene bound cyano borohydride

rt=Room temperature

SiO₂=Silica gel

THF=Tetrahydrofuran

### Intermediates

3-((1R,3R,5S)-8-Methyl-8-aza-bicyclo[3.2.1]oct-3-yloxy)-benzonitrile hydrochloride.

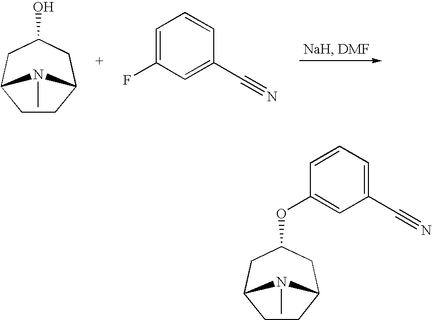

To a stirred solution of tropine hydrate (0.582 g, 4.1 mmol) in DMF (5 mL) was added NaH (0.2 g, 6.15 mmol of 60% mineral oil suspension) and the mixture stirred for ten minutes. To this was added 3-fluorobenzenenitrile (0.50 g, 4.1 mmol) and the resultant slurry heated to 100° C. for 1 hour. The material was then partitioned between ethyl acetate (70 mL) and H₂O (100 mL), and the organic layer was collected. The ethyl acetate layer was washed with brine (1×50 mL) and dried over Na₂SO₄. The material was filtered and concentrated to give the title compound as a colorless oil. The oil was dissolved in diethyl ether and treated with 1 N HCl/Et₂O to afford the hydrochloride salt after filtration (0.40 g, 35%).

¹H NMR (DMSO-d₆) δ 1.91-1.96 (m, 2H), 2.23 (br s, 4H), 2.42-2.47 (m, 2H), 2.70 (s, 3H), 3.87 (br s, 2H), 4.74-4.80 (br s, 1H), 7.33 (dd, 1H, J=1.8 Hz, 7.5 Hz), 7.41 (d, 1H, J=7.5 Hz), 7.51-7.60 (m, 2H).

3-(1-Methyl-piperidin-4-yloxy)-benzonitrile hydrochloride

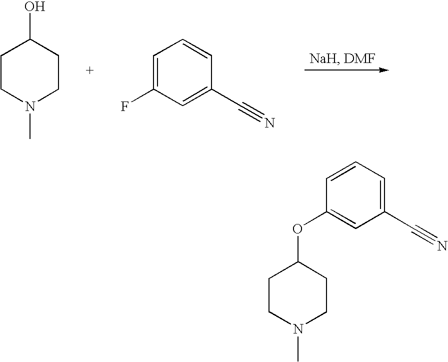

An analogous procedure was followed to that used to produce 3-((1R,3R,5S)-8-methyl-8-aza-bicyclo[3.2.1]oct-3-yloxy)-benzonitrile hydrochloride to give the title compound as a white solid (0.900 g, 27%).

¹H NMR (DMSO-d₆) δ 1.58-1.68 (m, 2H), 1.89-1.98 (m, 2H), 2.18-2.21 (m, 2H), 2.59-2.62 (m, 2H), 3.28 (s, 3H), 4.44-4.51 (m, 1H), 7.29 (dd, 1H, J=1.8, 8.1 Hz), 7.37 (d, 1H, J=8.1 Hz), 7.43-7.49 (m, 2H). The material can be treated with PS-CO₃²⁻ (3 equiv) in CH₂Cl₂ for 3 h, filtered and concentrated to give the free base.

3-((1R,3R,5S)-8-Methyl-8-aza-bicyclo[3.2.1]oct-3-yloxy)-benzylamine dihydrochloride

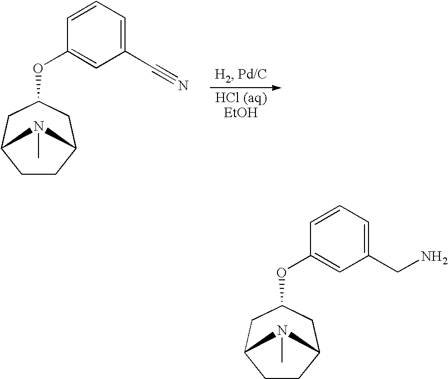

A solution of 3-((1R,3R,5S)-8-methyl-8-aza-bicyclo[3.2.1]oct-3-yloxy)-benzonitrile hydrochloride (400 mg) was dissolved in EtOH and treated with 10% Pd/C (˜200 mg), followed by conc. aq. HCl (0.1 mL). The material was shaken at 40 psi of hydrogen pressure for 6 h, filtered and concentrated to dryness. The resultant residue was washed with diethyl ether and used as is without further purification.

¹H NMR (DMSO-d₆) δ 2.07-2.12 (m, 2H), 2.38 (br s, 4H), 2.55-2.59 (m, 2H), 2.68 (s, 3H), 3.86 (br s, 2H), 3.99 (br s, 2H), 4.70 (br s, 1H, 6.95 (dd, 1H, J=1.8 Hz, 7.8 Hz), 7.07 (d, 1H, J=7.2 Hz), 7.16 (br s, 1H), 7.34 (t, 1H, J=7.8 Hz), 8.44 (br s, 2H).

3-(1-Methyl-piperidin-4-yloxy)-benzylamine hydrochloride

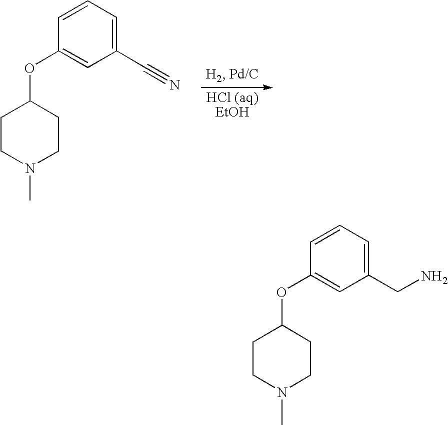

The title compound was prepared in an analogous fashion to 3-((1R,3R,5S)-8-methyl-8-aza-bicyclo[3.2.1]oct-3-yloxy)-benzylamine dihydrochloride (0.032 g, 15%).

¹H NMR (DMSO-d₆) δ 1.56-1.67 (m, 2H); 1.86-1.95 (m, 2H); 2.15-2.19 (m, 5H); 2.51-2.63 (m, 2H); 3.66 (s, 2H); 4.28-4.37 (m, 1H); 6.75 (dd, 1H, J=2.1, 8.1 Hz); 6.84 (d, 1H, J=7.5 Hz); 6.91 (s, 1H); 7.17 (t, 1H, J=7.8 Hz).

Methyl-[3-(1-methyl-piperidin-4-yloxy)-benzyl]-amine

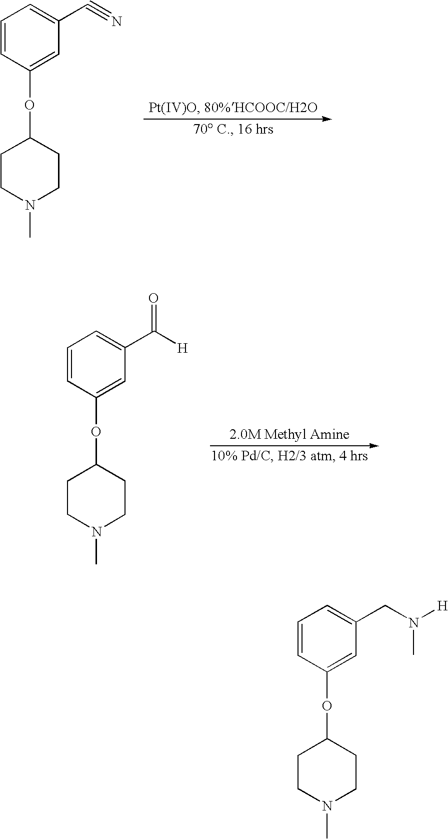

3-(1-Methyl-piperdin-4-yloxy)-benzonitrile (5 g, 23.1 mmol) was dissolved in an 80% solution of formic acid/H₂O. Pt(IV)O (0.524 g, 2.31 mmol) was added and the reaction mixture was allowed to stir and heat at 70° C. for 16 h. Next, the reaction was filtered and fresh Pt(IV)O (0.262 g, 1.15 mmol) was added. The reaction was allowed to continue stirring and heating for an additional 4 hours. LC/MS monitoring of the reaction indicated reaction completion at this point. The reaction mixture was filtered and the formic acid solution removed via rotary evaporation. The residual light-yellow semi-solid was dissolved in methylene chloride and washed with saturated NaHCO₃, H₂O, and brine. The organic layer was dried over MgSO₄ and concentrated to yield 3.95 gm of the corresponding aldehyde 3-(1-methyl-piperidin-4-yloxy)-benzaldehyde, (77%). LC/MS [M+H]⁺ calculated: 220.29, found: 220.2. ¹H NMR (300 MHz, CDCl₃) δ 9.98 (s, 1H), 7.51 (m, 1H), 7.41 (d, 1H), 7.20 (m, 2H), 4.76 (m, 1H), 3.30 (m, 4H), 2.81 (s, 3H), 2.62 (m, 2H), 2.23 (m, 2H). Product was used without further purification. The aldehyde was dissolved in 50 ml of 2.0M methyl amine in methanol. A catalytic amount of 10% Pd/C was added and mixture hydrogenated for 4 h at 3 atm. LC/MS monitoring of reaction indicated reaction completion. Reaction mixture was filtered and concentrated. Purification via silica gel chromatography using (9/0.9/0.1) mixture of (CH₂Cl₂/CH₃OH/NH₃OH) afforded 4.0 g of methyl-[3-(1-methyl-piperidin-4-yloxy)-benzyl]-amine, (90%).

¹H NMR (300 MHz, CDCl₃, 300K) δ 7.25 (t, 1H), 7.06 (s, 1H), 6.98 (d, 1H), 6.86 (d, 1H), 5.06 (s, 1H), 4.40 (m, 1H), 3.87 (d, 2H), 2.82 (m, 2H), 2.49 (m, 5H), 2.40 (s, 3H), 2.14 (m, 2H), 1.91 (m, 2H).

Methyl-[3-((1S, 3R, 5R)-8-methyl-8-aza-bicyclo[3.2.1]oct-3-yloxy)-benzyl]-amine.

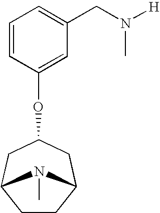

Prepared according to the method described for methyl-[3-(1-methyl-piperidin-4-yloxy)-benzyl]-amine.

¹H NMR (300 MHz, CDCl₃) δ 7.21 (t, 1H), 6.83 (d, 1H), 6.81 (s, 1H), 6.86 (d, 1H), 6.71 (d, 1H), 4.53 (m, 1H), 3.71 (s, 2H), 3.11 (bs, 1H), 2.46 (s, 3H), 2.30 (s, 3H), 2.07 (m, 11H).

4-Bromo-N-[3-(1-methyl-piperidin-4-yloxy)-benzyl]-benzamide hydrochloride salt

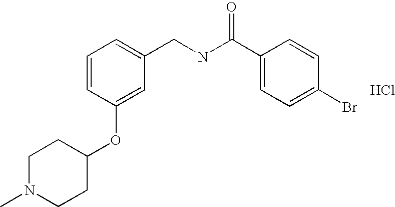

A solution of 4-bromobenzoyl chloride (0.29 g, 1.3 mmol) in acetonitrile (5 mL) was added to a stirred solution of 3-(1-methyl-piperidin-4-yloxy)-benzylamine (0.29 g, 1.3 mmol) in acetonitrile (25 mL) and allowed to react 18 h. The reaction mixture was evaporated to a solid and used without further purification.

¹H NMR (300.132 MHz, DMSO-d6) δ 10.45-10.26 (m, 1H), 9.10 (t, J=5.8 Hz, 1H), 7.85 (d, J=8.4 Hz, 2H), 7.69 (d, J=8.5 Hz, 2H), 7.30-7.21 (m, 1H), 7.00-6.83 (m, 3H), 4.75-4.40 (m, 3H), 3.52-3.01 (m, 7H), 2.28-1.72 (m, 4H).

4-Bromo-N-[3-((1S,3R,5R)-8-methyl-8-aza-bicyclo[3.2.1]oct-3-yloxy)-benzyl]-benzamide hydrochloride salt

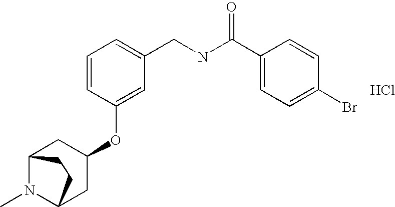

The title compound was prepared as described for 4-bromo-N-[3-(1-methyl-piperidin-4-yloxy)-benzyl]-benzamide hydrochloride salt as a solid. This compound was used without further characterization.

3-Bromo-N-[3-(1-methyl-piperidin-4-yloxy)-benzyl]-benzamide hydrochloride salt

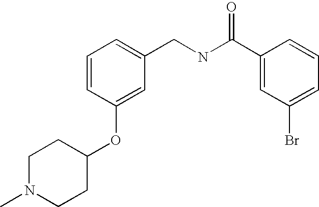

The title compound was prepared as described for 4-bromo-N-[3-(1-methyl-piperidin-4-yloxy)-benzyl]-benzamide hydrochloride salt as a solid.

NMR of free base ¹H NMR (300.132 MHz, CDCl₃) δ 7.94-7.91 (m, 1H), 7.73-7.60 (m, 2H), 7.34-7.22 (m, 3H), 6.94-6.81 (m, 3H), 6.37-6.26 (m, 1H), 4.59 (d, J=5.6 Hz, 2H), 4.33 (dquintet, J=7.5, 3.7 Hz, 1H), 2.75-2.64 (m, 2H), 2.37-2.23 (m, 5H), 2.08-1.95 (m, 2H), 1.90-1.77 (m, 2H)

### Exemplary Compounds

### EXAMPLE 1

### N-[3-((1R,3R,5S)-8-Methyl-8-aza-bicyclo[3.2.1]oct-3-yloxy)-benzyl]-4-phenoxy-benzamide hydrochloride

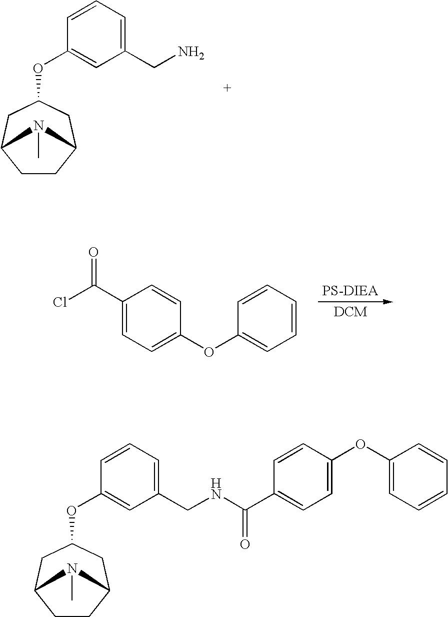

To a solution of 3-((1R,3R,5S)-8-methyl-8-aza-bicyclo[3.2.1]oct-3-yloxy)benzylamine dihydrochloride amine (0.100 g, 0.31 mmol, 1.5 equiv) in methylene chloride (1 mL) was added PS-DIEA (3 equiv, 3.88 mmol/g), followed by 4-phenoxy-benzoyl chloride (0.10 g, 0.20 mmol, 1.0 equiv). The resultant suspension was stirred for 4 h, filtered and chromatographed (SiO₂, using a gradient of 100% CH₂Cl₂ to 95/5 CH₂Cl₂/2N NH₃ in MeOH) to give a gummy residue. The material was dissolved in diethyl ether and converted to the hydrochloride salt by treatment with 1N HCl in diethyl ether. The resultant solid was collected by filtration and dried to give the title compound as a white solid. (0.05 g, 36%).

¹H NMR (DMSO-d₆) δ TFA shake 1.90-1.95 (m, 2H), 2.23 (br s 4H), 2.34-2.39 (m, 2H), 2.70 (s, 3H), 3.86 (m, 3H), 4.44 (m, 2H), 4.68 (br m, 1H), 6.84 (d, 1H, J=8.7 Hz), 6.90-6.93 (m, 2H), 7.04 (d, 2H, J=8.7 Hz), 7.08 (d, 2H, J=7.5 Hz), 7.18-7.28 (m, 2H), 7.34-7.51 (m, 2H), 7.91 (d, 2H, J=8.7 Hz).

### EXAMPLE 2

### N-[3-((1R,3R,5S)-8-Methyl-8-aza-bicyclo[3.2.1]oct-3-yloxy)-benzyl]-4-propyl-benzamide

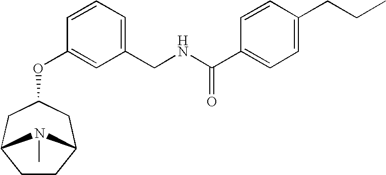

The title compound was prepared in analogous fashion to N-[3-((1R,3R,5S)-8-methyl-8-aza-bicyclo[3.2.1]oct-3-yloxy)-benzyl]-4-phenoxy-benzamide to give the title compound as a white solid (0.008 g, 40%).

¹H NMR (DMSO-d₆) δ 0.92 (t, 3H, J=7.5 Hz), 1.60 (m, 2H), 2.10 (m, 2H), 2.24 (br s, 2H), 2.32 (m, 2H), 2.61 (t, 2H, J=7.5 Hz), 2.71 (s, 3H), 3.87 (m, 3H), 4.45 (m, 2H), 4.68 (m, 1H), 6.84 (d, 1H, J=9.0 Hz), 6.91-6.94 (m, 2H), 7.23-7.30 (m, 4H), 7.81 (d, 1H, J=8.1 Hz).

### EXAMPLE 3

### 4-Cyclohexyl-N-[3-((1R,3R,5S)-8-methyl-8-aza-bicyclo[3.2.1]oct-3-yloxy)-benzyl]-benzamide

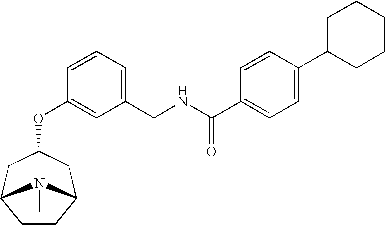

The title compound was prepared in analogous fashion to N-[3-((1R,3R,5S)-8-methyl-8-aza-bicyclo[3.2.1]oct-3-yloxy)-benzyl]-4-phenoxy-benzamide to give the title compound as a white solid (0.012 g, 20%).

¹H NMR (DMSO-d₆) δ 1.23-2.15 (m, 18H), 2.30 (s, 2H), 2.54-2.60 (m, 1H), 3.12 (m, 2H), 3.48 (s, 3H), 4.51 (t, 1H, J=5.1 Hz), 4.59 (d, 2H, J=5.1 Hz), 6.29 (m, 1H), 6.73 (d, 1H, J=8.4 Hz), 6.81 (s, 1H), 6.89 (d, 1H, J=7.5 Hz), 7.21-7.27 (m, 3H), 7.70 (d, 2H, J=8.4 Hz).

### EXAMPLE 4

### 4-Benzyl-N-[3-(1-methyl-piperidin-4-yloxy)-benzyl]-benzamide

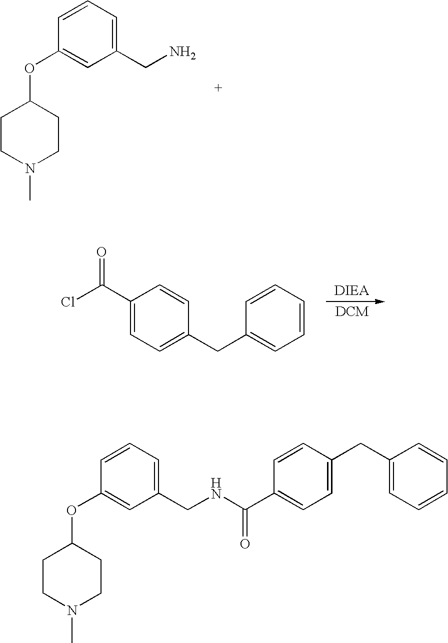

To a solution of 3-(1-methyl-piperidin-4-yloxy)-benzylamine (66 mg, 300 μmol) in CH₂Cl₂ (6 mL) was added freshly prepared 4-benzyl-benzoyl chloride (300 μmol, obtained from the reaction of 4-benzyl-benzoyl acid and oxalyl chloride) and diethylamine (1 mL, 570 μmol) and this mixture was stirred at rt overnight. The mixture was concentrated and partitioned between CH₂Cl₂ and 1M NaOH; the layers were separated, the aqueous washed with additional CH₂Cl₂, the organic phases were combined and concentrated to afford the title compound as a solid (41 mg, 34%).

¹H NMR(DMSO-d₆) δ 1.55-1.66 (m, 2H); 1.82-1.92 (m, 2H); 2.15-2.17 (m, 5H); 2.56-2.62 (m, 2H); 3.99 (s, 2H); 4.29-4.33 (m, 1H); 4.41 (d, 2H, J=6 Hz); 6.79-6.85 (m, 3H); 7.17-7.33 (m, 8H); 7.80 (d, 2H, J=8.1 Hz); 8.89 (t, 1H, J=6 Hz).

### EXAMPLE 5

### N-[3-(1-Methyl-piperidin-4-yloxy)-benzyl]-3-phenoxy-benzamide

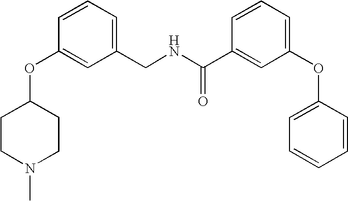

The title compound was prepared in analogous fashion to 4-benzyl-N-[3-(1-methyl-piperidin-4-yloxy)-benzyl]-benzamide to give the title compound (70 mg, 58%).

¹H NMR (DMSO-d₆) δ 1.55-1.66 (m, 2H); 1.85-1.90 (m, 2H); 2.10-2.16 (m, 5H); 2.57-2.61 (m, 2H); 4.27-4.33 (m, 1H); 4.41 (d, 2H, J=5.97 Hz); 6.79-6.85 (m, 3H); 7.04 (dd, 2H, J=1.1, 8.7 Hz); 7.14-7.23 (m, 3H); 7.38-7.52 (m, 4H); 7.67 (d, 1H, J=8 Hz); 9.02 (t, 1H, J=5.94 Hz).

### EXAMPLE 6

### N-[3-(1-Methyl-piperidin-4-yloxy)-benzyl]-4-phenoxy-benzamide

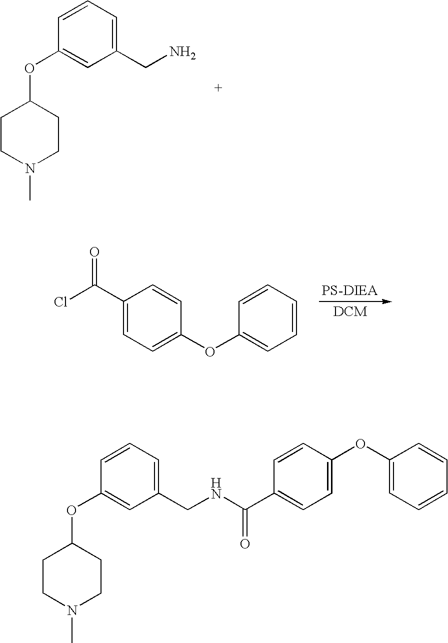

To a solution of 4-benzyloxybenzoyl chloride (0.04 g, 0.18 mmol) in DCM was added 3-(1-methyl-piperidin-4-yloxy)-benzylamine (0.04 g, 0.18 mmol). The reaction was stirred for 3 h, filtered and then chromatographed (SiO₂, CH₂Cl₂ to 5% gradient of 2N NH₃ in MeOH). The resultant material was treated with 1 N HCl in Et₂O to give the title compound (0.04 g, 50%).

¹H NMR (DMSO-d₆) δ TFA shake 1.75-1.82 (m, 1H), 2.04 (m, 2H), 2.22-2.26 (m ,1H), 2.79-2.81 (app d, 3H), 3.00-3.19 (m, 2H), 3.30-3.37 (m ,2 H), 3.42-3.51 (m, 2H), 4.44-4.51 (m, 3), 6.87-6.97 (m, 3H), 7.03-7.10 (m, 4H), 7.44 (t, 2H, J=7.5 Hz), 7.92 (d, 2H, J=8.7 Hz).

### EXAMPLE 7

### -Benzyloxy-N-[3-(1-methyl-piperidin-4-yloxy)-benzyl]-benzamide

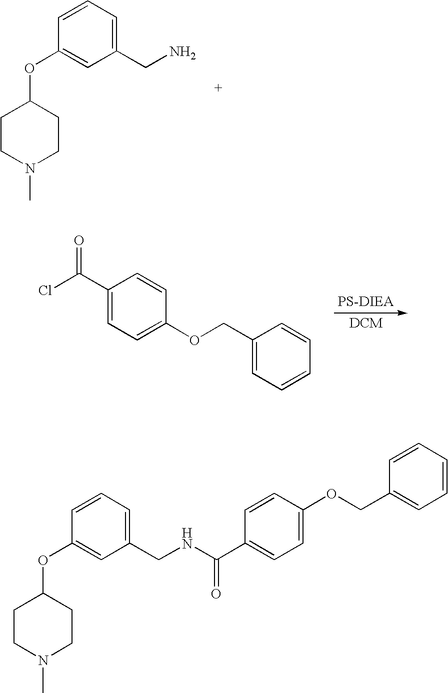

The title compound was prepared in analogous fashion to N-[3-(1-methyl-piperidin-4-yloxy)-benzyl]-4-phenoxy-benzamide to give a white solid (0.028 g, 35%).

¹H NMR (CDCl, 300 MHz) δ 1.59-1.62 (m, 2H), 1.87-1.89 (m, 2H), 2.18 (m, 3H), 2.59-2.61 (m, 2H), 4.30 (m, 1H), 4.40 (m, 2H), 5.17 (m, 2H), 6.79 (m, 3H), 7.06-7.09 (m, 2H), 7.20-7.22 (br m, 1H), 7.33-7.47 (m 5H), 7.85 (d, 2H), 8.87 (s, 1H).

### EXAMPLE 8

### Biphenyl-4-carboxylic acid 3-(1-methyl-piperidin-4-yloxy)-benzylamide

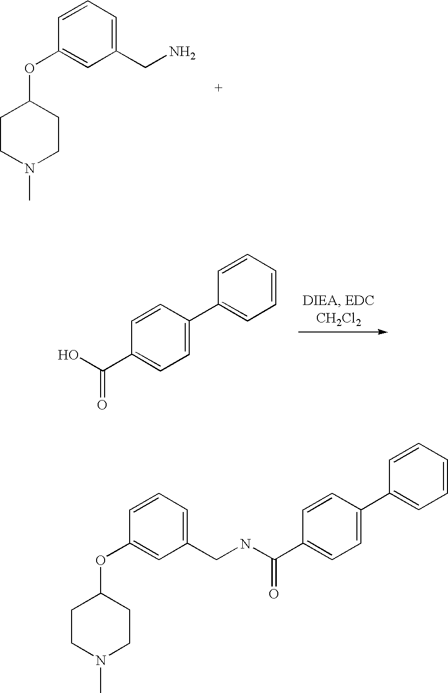

To a solution of 3-(1-methyl-piperidin-4-yloxy)-benzylamine (100 mg, 456 μmol) in CH₂Cl₂ (5 mL) was added 4-phenyl benzoic acid (456 μmol), DIEA (156 μL, 900 μmol) and EDC (171 mg, 456 μmol). The reaction was stirred at room temperature until completed; solvent was removed, residue was dissolved in ethyl acetate, washed with brine, dried over MgSO₄ and filtered and concentrated. Purification by column chromatography (SiO₂; 0-8% CH₂Cl₂—CH₂Cl₂/1% NH₄OH in MeOH) afforded the title compound (100 mg, 250 μmol, 56%) as a solid.

¹H NMR (CDCl₃, 300 MHz) δ 1.80-1.89 (m, 2H); 1.82-2.1 (m, 2H); 2.3-2.4 (m, 2H); 2.30 (s, 3H); 2.6-2.72 (m, 2H); 4.28-4.32 (m, 1H); 4.63 (d, 2H, J=5.7 Hz); 6.46 (m, 1H); 6.84 (d, 1H, J=9 Hz); 6.92-6.94 (m, 1H); 7.23-7.28 (m, 2H); 7.37-7.48 (m, 3H); 7.60 (d, 2H, J=7.2 Hz); 7.65 (d, 2H, J=8.1 Hz); 7.86 (d, 2H, J=8.1 Hz).

### EXAMPLE 9

### N-[3-(1-Benzyl-piperidin-4-yloxy)-benzyl]-4-phenoxy-benzamide

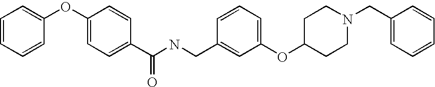

Prepared as in 4-phenoxy-N-[3-(1-ethyl-piperidin-4-yloxy)-benzyl]-benzamide to give the title compound as a white solid.

¹H NMR (CDCl₃, 300 MHz) δ 1.25 (t, 2H, J=7.14 Hz), 1.82 (m, 2H), 1.96 (m, 2H), 2.30 (m, 2H), 2.73 (m, 2H), 3.53 (s, 2H), 4.31 (m, 1H), 4.59 (d, 2H, J=5.5 Hz), 6.26 (m, 1H), 6.82 (dd, 1H, J=7.2, 1.5 Hz), 6.90 (m, 2H), 7.00 (dd, 2H, 7.0, 1.5 Hz), 7.03 (d, 2H, J=7.5 Hz), 7.16 (t, 1H, J=7.4 Hz), 7.23-7.39 (m, 8H), 7.75 (d, 2H, J=7.0 Hz).

### EXAMPLE 10

### 4-Phenoxy-N-[3-(piperidin-4-yloxy)-benzyl]-benzamide

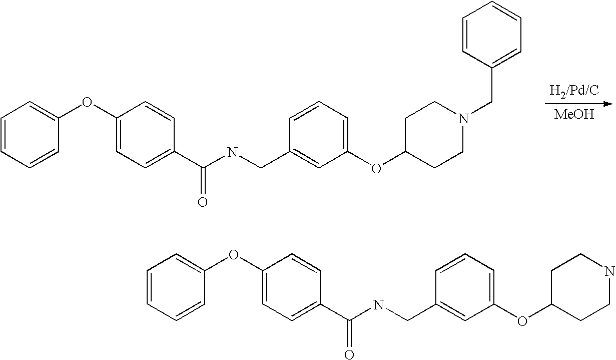

To a solution of N-[3-(1-benzyl-piperidin-4-yloxy)-benzyl]-4-phenoxy-benzamide in MeOH was added Pd/C (10%) and the mixture was agitated under 40 psi of H₂ for 12 h. Filtration gave the material as a crude oily material.

¹H NMR (DMSO-d6, 300 MHz) δ 1.36-1.43 (m, 2H), 1.86-1.90 (m, 2H), 2.50-2.57 (m, 2H), 2.90-2.94 (m, 2H), 4.30 (m, 1H), 4.42 (d, 2H, J=5.8 Hz), 6.79-6.85 (m, 3H), 7.03 (d, 2H, 8.5 Hz), 7.07 (d, 2H, J=7.6 Hz), 7.20 (t, 2H, J=7.5 Hz), 7.43 (t, 2H, J=7.6 Hz), 7.91 (d, 2H, J=8.7 Hz), 8.92 (t, 1H, J=5.8 Hz).

### EXAMPLE 11

### 4-Phenoxy-N-[3-(1-ethyl-piperidin-4-yloxy)-benzyl]-benzamide

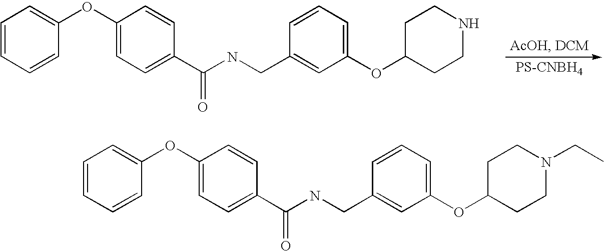

To a stirred solution of 4-phenoxy-N-[3-(piperidin-4-yloxy)-benzyl]-benzamide (7.5 mmol, 0.03 g) in dichloromethane (2 mL) was added acetaldehyde (9.5 mmol), acetic acid (0.3 mL) and PS-CNBH₄ (ca. 100 mg, 2.57 mmol/g loading). The reaction was stirred for one hour, filtered and chromatographed (SiO₂, CH₂Cl₂/MeOH gradient 100% to 90%/10%). The isolated fractions were collected and the solvent removed. The residual material was dissolved in dichloromethane, and washed with sat. NaHCO₃.

¹H NMR (CDCl₃, 300 MHz) δ 1.09 (t, 3H, J=7.2 Hz), 1.76-1.85 (m, 2H), 1.97-2.02 (m, 2H), 2.24-2.30 (m, 2H), 2.41 (q, 2J, J=7.2 Hz), 2.61-2.73 (m, 2H), 4.29-4.34 (m, 1H), 4.59 (d, 2H, J=5.5 Hz), 6.28-6.39 (m, 1H), 6.81 (dd, 1H, J=1.5, 7.2 Hz), 6.81-6.92 (m, 2H), 7.00 (d, 2H, J=8.7 Hz), 7.03 (d, 2H, J=8.3 Hz), 7.16 (t, 1H, J=7.5 Hz), 7.24-7.27 (m, 1H), 7.37 (t, 2H, J=8.2 Hz), 7.75 (d, 2H, J=8.7 Hz).

### EXAMPLE 12

### 4-Phenoxy-N-[3-(1-propyl-piperidin-4-yloxy)-benzyl]-benzamide

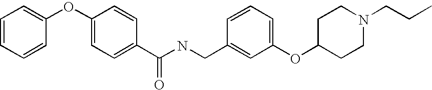

Prepared in an analogous fashion to 4-phenoxy-N-[3-(1-ethyl-piperidin-4-yloxy)-benzyl]-benzamide to give the title compound as a white solid.

¹H NMR (CDCl₃, 300 MHz) δ 0.92 (t, 3H, J=7.3 Hz), 1.58 (m, 2H), 1.89 (m, 2), 2.10 (m, 2H), 2.42 (m, 2H), 2.81 (m, 2H), 4.38 (m, 1H), 4.60 (d, 2H, J=5.6 Hz), 6.28 (m, 1H), 6.83 (d, 1H, J=8.4 Hz), 6.91 (m, 2H), 7.00 (d, 2H, J=8.7 Hz), 7.03 (d, 2H, J=7.6 Hz), 7.16 (t, 1H, J=7.4 Hz), 7.25 (m, 4H), 7.37 (t, 2H, J=8.2 Hz), 7.75 (d, 2H, J=8.7 Hz).

### EXAMPLE 13

### N-Ethyl-N-[3-(1-ethyl-piperidin-4-yloxy)-benzyl]-4-phenoxy-benzamide

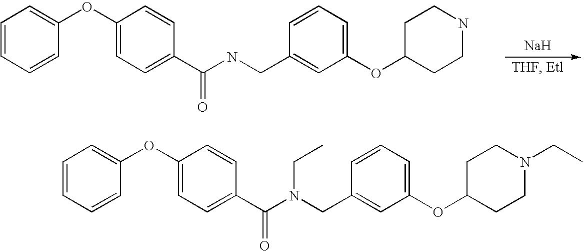

To a stirred solution of 4-phenoxy-N-[3-(piperidin-4-yloxy)-benzyl]-benzamide (0.12 mmol, 0.05 g) in THF was added NaH (0.15 mmol, 60% in mineral oil, 0.006 g) and the reaction stirred for 20 min. To this was added ethyl iodide (0.3 mmol) and the reaction stirred for 3 h. The mixture was partitioned between ethyl acetate and H₂O, the organics evaporated and then collected. The material was purified by chromatography (SiO₂, CH₂Cl₂ to 10% MeOH gradient) to give the title compound as a colorless oil.

¹H NMR (CDCl₃, 300 MHz) δ 1.14 (t, 3H, J=7.2 Hz), 1.25 (m, 3H), 1.97 (m, 2H), 2.24 (m, 2H), 2.67 (m, 2H), 2.89 (m, 2H), 3.38 (m, 2H), 4.43 (m, 1H), 4.61 (s, 2H), 6.79-6.89 (m, 3H), 6.99-7.03 (m, 4H), 7.12 (t, 1H, J=7.2 Hz), 7.21 (m, 1H), 7.31-7.41 (m, 4H).

### EXAMPLE 14

### [3-(1-Methyl-piperidin-4-yloxy)-benzyl]-(4-phenoxy-benzyl)-amine

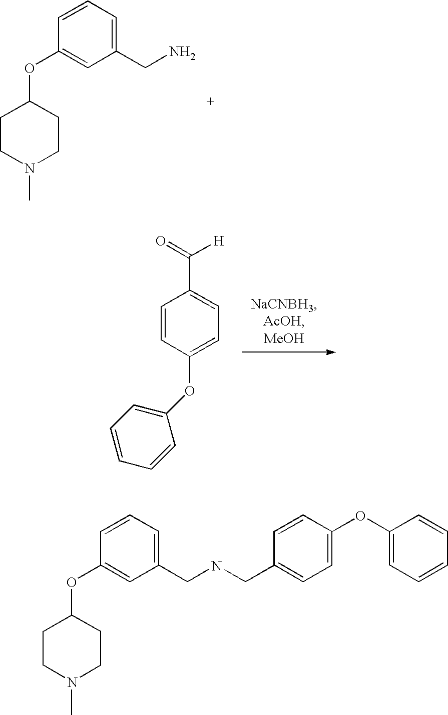

To a solution of 3-(1-methyl-piperidin-4-yloxy)-benzylamine (66 mg, 300 μmol) in MeOH (2 mL) was added 5-phenoxybenzaldehyde (60 mg, 300 μmol) followed by sodium cyanoborohydride (27 mg, 400 μmol) and acetic acid (2 drops). This mixture was allowed to stir at rt overnight, at which point it was concentrated and partitioned between CH₂Cl₂ and 1M NaOH. The phases were separated and the aqueous phase extracted with additional CH₂Cl₂; the organic phases were concentrated and purified by column chromatography (SiO₂; 0-8% CH₂Cl₂—CH₂Cl₂/1% NH₄OH in MeOH). This afforded the title compound as an oil (63 mg, 52%).

¹H NMR (DMSO-d6, 300 MHz) δ 1.56-1.67 (m, 2H); 1.75-1.90 (m, 2H); 2.12-2.19 (m, 5H); 2.58-2.62 (m, 2H); 3.63 (app S, 4H); 4.29-4.37 (m, 1H); 6.79 (dd, 1H, J=17.7, 8.1 Hz); 6.87 (d, 1H, J=7.5 Hz); 6.94-6.99 (m, 5H); 7.12 (t, 1H, J=7.5 Hz); 7.19 (t, 1H, J=7.8 Hz); 7.33-7.40 (m, 4H).

### EXAMPLE 15

### (4-Isopropyl-benzyl)-[3-(1-methyl-piperidin-4-yloxy)-benzyl]-amine

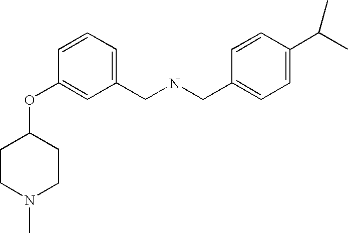

Prepared in an analogous fashion to [3-(1-methyl-piperidin-4-yloxy)-benzyl]-(4-phenoxy-benzyl)-amine to give the title compound (11 mg, 10%).

¹H NMR (CDCl₃, 300 MHz) δ 1.24 (s, 6H); 1.79-1.90 (m, 2H); 1.97-2.03 (m, 2H); 2.25-2.30 (m, 2H); 2.31 (s, 3H); 2.65-2.71 (m, 2H); 2.85-2.94 (m, 1H); 3.77 (s, 2H); 3.78 (s, 2H); 4.30-4.35 (m, 1H); 6.79 (dd, 1H, J=8.7, 1.8 Hz); 6.89-6.92 (m, 2H); 7.15-7.31 (m, 5H).

### EXAMPLE 16

### Benzo[1,3]dioxol-5-ylmethyl-[3-(1-methyl-piperidin-4-yloxy)-benzyl]-amine

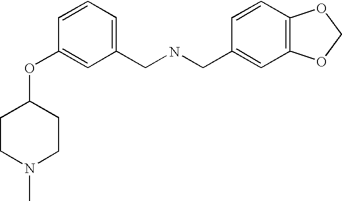

Prepared in an analogous fashion to [3-(1-methyl-piperidin-4-yloxy)-benzyl]-(4-phenoxy-benzyl)-amine to give the title compound (29 mg, 26%).

¹H NMR (CDCl₃, 300 MHz) δ 1.78-1.89 (m, 2H); 1.96-2.03 (m, 2H); 2.24-2.29 (m, 2H); 2.30 (s, 3H); 2.65-2.69 (m, 2H); 3.71 (s, 2H); 3.74 (s, 2H); 4.29-4.34 (m, 1H); 5.90 (s, 2H); 6.73-6.80 (m, 3H); 6.86-6.90 (m, 3H); 7.18-7.24 (m, 1H).

### EXAMPLE 17

### (4-Chloro-benzyl)-[3-(1-methyl-piperidin-4-yloxy)-benzyl]-amine

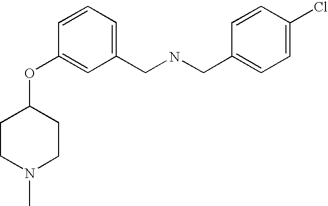

Prepared in an analogous fashion to [3-(1-methyl-piperidin-4-yloxy)-benzyl]-(4-phenoxy-benzyl)-amine to give the title compound (6 mg, 6%).

¹H NMR (CDCl₃, 300 MHz) δ 1.88-1.99 (m, 2H); 2.04-2.15 (m, 2H); 2.46 (s, 3H); 2.5-2.6 (m, 2H); 2.8-2.88 (m, 2H); 3.49-3.5 (m, 4H); 4.35-4.4 (m, 1H); 6.76-6.79 (m, 1H); 6.89 (m, 1H); 6.94 (d, 1H, J=7.8 Hz); 7.19-7.28 (m, 5H).

### EXAMPLE 18

### Methyl-[3-(1-methyl-piperidin-4-yloxy)-benzyl]-(4-phenoxy-benzyl)-amine

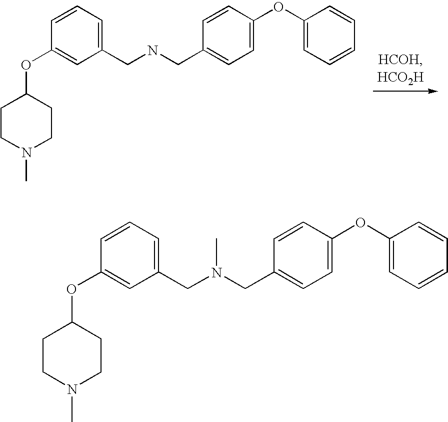

A mixture of [3-(1-methyl-piperidin-4-yloxy)-benzyl]-(4-phenoxy-benzyl)-amine (100 mg, 248 μmol) in formic acid (2 mL) and formaldehyde (10 mL) was refluxed overnight. The reaction was cooled, solvent removed in vacuo and the residue was purified by column chromatography (SiO₂, 10% MeOH in CH₂Cl₂). This afforded the title compound (31 mg, 30%).

¹H NMR (CDCL₃, 300 MHz) δ 1.79-1.88 (m, 2H); 1.96-1.98 (m, 2H); 2.19-2.30 (m, 2H); 2.29 (s, 6H); 2.67-2.72 (m, 2H); 3.53 (app s, 4H); 4.26-4.31 (m, 1H); 6.77 (dd, 1H, J=1.5, 9 Hz); 6.93-7.0 (m, 6H); 7.08 (t, 1H, J=7.5 Hz); 7.20-7.33 (m, 5H).

### EXAMPLE 19

### N-[3-(4-Methyl-piperazin-1-ylmethyl)-benzyl]-4-phenoxy-benzamide

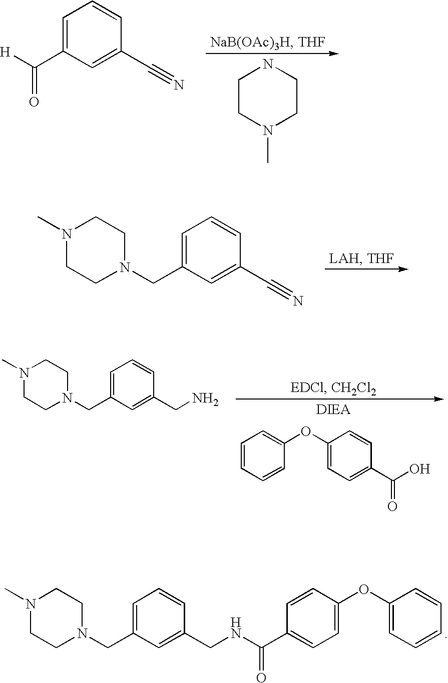

To a stirred solution of 3-cyanobenzaldehyde (3.25 mmol) was added N-methyl piperizine (3.25 mmol) followed by NaB(OAc)₃H (4.25 (mmol). The mixture was stirred for 4 h and concentrated. The residual material was partitioned between CH₂Cl₂ and NaHCO₃ (sat.) and the organic phase was then collected. The organic phase was concentrated (0.70 g, 3.25 mmol) and dissolved in THF (5 mL). The solution was cooled in an ice bath, and to this was added lithium aluminum hydride (1.0 M in THF, 4.8 mL, 4.88 mmol). The solution was warmed to room temperature and stirred for 2 h. The reaction was quenched with excess sodium sulfate decahydrate (˜1 g), and then filtered through diatomaceous earth. The material was concentrated to give a clear oil of the benzylamine which was used without further purification. 4-Phenoxy benzoic acid was dissolved in CH₂Cl₂ (2 mL) and to this was added EDCI (0.107 g, 0.56 mmol) followed by DIEA (0.048 mL, 0.56 mmol) and the benzyl amine obtained from the previous step (0.10 g, 0.50 mmol). The reaction was stirred for 2 h, and then partitioned between CH₂Cl₂ and NaHCO₃ (sat.). The organic layer was concentrated and the residual oil chromatographed (SiO₂, CH₂Cl₂/5% NH₃ in MeOH, 95:5) to give the title compound as a white solid.

¹H NMR (CDCl₃, 300 MHz) δ 2.30 (s, 3H), 2.49 (br s, 8H), 3.51 (s, 2H), 4.63 (d, 2H, J=5.4 Hz), 6.27 (s, 1H), 6.96-7.05 (m, 4H), 7.16 (t, 1H, J=7.5 Hz), 7.25 (m, 1H), 7.30-7.39 (m, 5H), 7.75-7.78 (d, 2H, J=7.8 Hz).

### EXAMPLE 20

### 4′-Methoxy-biphenyl-4-carboxylic acid methyl-[3-(1-methyl-piperidin-4-yloxy)-benzyl]-amide

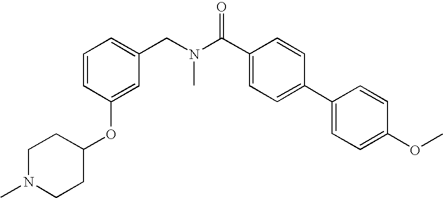

To a stirred solution of 4′-methoxy-biphenyl-4-carboxylic acid 3-(1-methyl-piperidin-4yloxy)-benzylamide (0.100 g, 0.2 mmol) in DMF (2 mL) was added NaH (0.02 g, 0.3 mmol, 60% mineral oil). The reaction was stirred for 10 minutes, and methyl iodide was added. The reaction was stirred for another 1 hour and then quenched with NaHCO₃. The reaction was partitioned between CH₂Cl₂ and H₂O and the organics concentrated. Chromatography (SiO2, CH₂Cl₂/10% MeOH 2N NH₃ gradient) gave the title compound.

¹H NMR (300 MHz, DMSO-d6) δ 7.63 (m, 4H), 7.45 (d, 2H, J=7.8 Hz), 7.26 (t, 1H, J=8.1 Hz), 7.05 (d, 2H, J=7.8 Hz), 6.85 (m, 3H), (4.33, m, 1H), 3.80 (s, 3H), 2.60 (m, 2H), 2.16 (s, 3H), 1.89 (m, 2H), 1.60 (m, 2H), 1.07 (m, 4H).

### EXAMPLE 21

### 4′-Methoxy-biphenyl-4-carboxylic acid methyl-[3-((1S,3R,5R)-8-methyl-8-aza-bicyclo[3.2.1]oct-3-yloxy)-benzyl]-amide

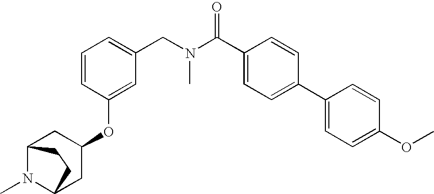

The title compound was prepared in a manner analogous to 4′-methoxy-biphenyl-4-carboxylic acid methyl-[3-(1-methyl-piperidin-4-yloxy)-benzyl]-amide, beginning with 4′-methoxy-biphenyl-4-carboxylic acid 3-((1S,3R,5R)-8-methyl-8-aza-bicyclo[3.2.1]oct-3-yloxy)-benzylamide.

¹H NMR (300 MHz, DMSO-d6) δ 7.6 (m, 4H), 7.47 (d, 2H, J=7.8 Hz), 7.28 (t, 1H, J=8.1 Hz), 7.05 (d, 2H, J=7.8 Hz), 6.85 (m, 3H), 4.55 (m, 3H), 3.80 (s, 3H), 3.07 (m, 2H), 2.89 (s, 3H), 2.21 (s, 3H), 2.10-1.89 (m, 8H).

### EXAMPLE 22

### 4′-Methoxy-biphenyl-4-carboxylic acid 3-((1S,3R,5R)-8-methyl-8-aza-bicyclo[3.2.1]oct-3-yloxy)-benzylamide

The title compound was prepared in a manner analogous to Example 4 to give a brown solid.

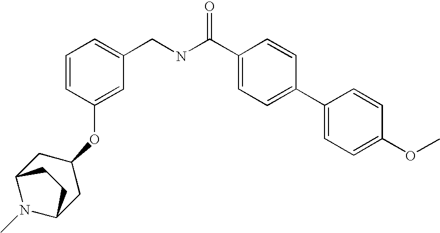

¹H NMR (300 MHz, DMSO-d6) δ 9.0 (t, 1H, J=6.0 Hz), 8.11 (d, 2H, J=8.7 Hz), 7.77 (m,4H), 7.22 (t, 1H, J=8.1 Hz), 7.00 (d, 2H, J=8.7 Hz), 6.92 (d, 1H, J=7.5 Hz), 6.80 (s, 1H), 6.70 (d, 1H, J=7.2 Hz), 4.52 (dt, 1H, J=5.1 Hz), 3.81 (s, 3H), 3.00 (m, 2H), 2.16 (s, 3H), 2.00-1.72 (m, 8H).

### EXAMPLE 23

### 4-Cyclohexyl-N-methyl-N-[3-(1-methyl-piperidin-4-yloxy)-benzyl]-benzamide

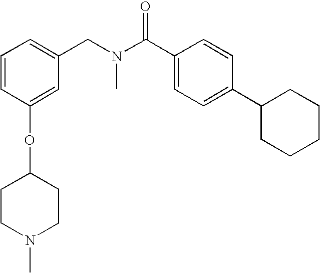

Prepared as described in Example 4 to give the title compound as a brown semi-solid.

¹H NMR (300.132 MHz, CDCl₃) δ 7.51-7.11 (m, 4H), 7.04-6.60 (m, 4H), 4.80-4.41 (m, 2H), 4.37-4.23 (m, 2H), 3.11-2.82 (m, 2H), 2.76-2.64 (m, 2H), 2.58-2.42 (m, 2H), 2.36-2.21 (m, 8H), 2.11-0.97 (m, 10H).

### EXAMPLE 24

### Biphenyl-4-ylmethyl-[3-(1-methyl-piperidin-4-yloxy)-benzyl]-amine

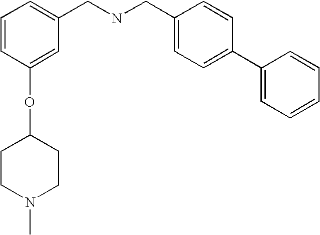

Prepared as described in Example 14 to give the title compound as a light brown semi-solid.

¹H NMR (300.132 MHz, CDCl₃) δ 7.67-6.72 (m, 13H), 4.48-4.21 (m, 1H), 3.82 (d, J=12.6 Hz, 4H), 2.82-2.63 (m, 2H), 2.43-2.27 (m, 5H), 2.17 (s, 1H), 2.08-1.96 (m, 2H), 1.94-1.79 (m, 2H)

### EXAMPLE 25

### Biphenyl-4-carboxylic acid methyl-[3-(1-methyl-piperidin-4-yloxy)-benzyl]-amide

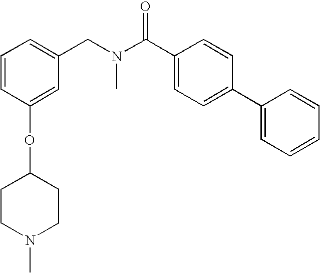

Prepared as described in Example 4 to give the title compound as a light-brown semi-solid.

¹H NMR (300.132 MHz, CDCl₃) δ 7.81-6.60 (m, 13H), 4.85-4.47 (m, 2H), 4.32 (s, 1H), 3.19-2.86 (m, 2H), 2.76-2.61 (m, 2H), 2.44-2.20 (m, 6H), 2.11-1.94 (m, 2H), 1.90-1.75 (m, 2H)

### EXAMPLE 26

### [3-((1S,3R,5R)-8-Methyl-8-aza-bicyclo[3.2.1]oct-3-yloxy)-benzyl]-(3-phenoxy-benzyl)-amine

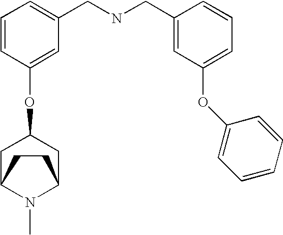

Prepared as described in Example 14 to give the title compound as light-brown semi-solid.

¹H NMR (300.132 MHz, CDCl₃) δ 7.44-6.56 (m, 13H), 4.61-4.43 (m, 1H), 3.76 (d, J=6.5 Hz, 4H), 3.18 (s, 1H), 2.35 (s, 3H), 2.28-1.83 (m, 10H)

### EXAMPLE 27

### Methyl-[3-((1S,3R,5R)-8-methyl-aza-bicyclo[3.2.1]oct-3-yloxy)-benzyl]-(4-phenoxy-benzyl)-amine

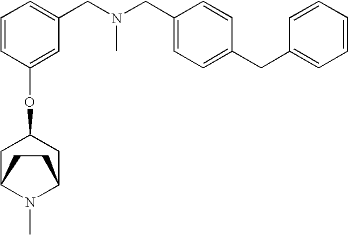

Prepared as described in Example 14 to give the title compound as a light-brown semi-solid.

¹H NMR (300.132 MHz, CDCl₃) δ 7.44-6.62 (m, 13H), 4.63-4.46 (m, 1H), 3.48 (d, J=4.9 Hz, 4H), 2.32 (s, 3H), 2.20 (s, 3H), 2.15-1.88 (m, 10H)

### EXAMPLE 28

### Biphenyl-4-ylmethyl-[3-((1S,3R,5R)-8-methyl-8-aza-bicylco[3.2.1]oct-3-yloxy)-benzyl]-amine

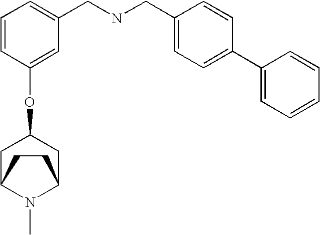

Prepared as described in Example 14 to give the title compound as a light-brown semi-solid.

¹H NMR (300.132 MHz, CDCl₃) δ 7.70-6.62 (m, 13H), 4.62-4.49 (m, 1H), 3.92-3.73 (m, 4H), 3.24 (s, 1H), 2.38 (s, 3H), 2.31-1.90 (m, 10H).

### EXAMPLE 29

### N-Methyl-N-[((1S,3R,5R)-8-methyl-8-aza-bicycl0[3.2.1]oct-3-yloxy)-benzyl]-3-phenoxy-benzamide

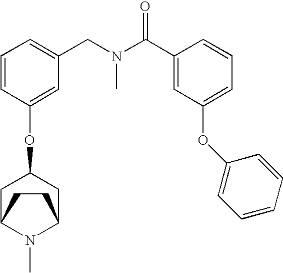

Prepared as described in Example 14 to give the title compound as a light brown semi-solid.

¹H NMR (300.132 MHz, CDCl₃) δ 7.56-6.46 (m, 13H), 4.68 (s, 2H), 4.60-4.28 (m, 1H), 2.29 (s, 6H), 2.18-1.83 (m, 10H).

### EXAMPLE 30

### Biphenyl-4-carboxylic acid 3-((1S,3R,5R)-8-methyl-8-aza-bicyclo[3.2.1]oct-3-yloxy)-benzamide

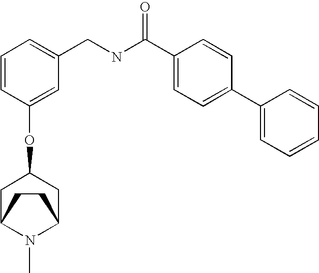

Prepared as described in Example 4 to give the title compound as a light brown semi-solid.

¹H NMR (300.132 MHz, CDCl₃) δ 7.98-6.34 (m, 13H), 4.62 (d, J=5.6 Hz, 2H), 4.56-4.46 (m, 1H), 2.29 (s, 3H), 2.19-1.81 (m, 10H)

### EXAMPLE 31

### N-Methyl-N-[((1S,3R,5R)-8-methyl-8-aza-bicycl0[3.2.1]oct-3-yloxy)-benzyl]-4-phenoxy-benzamide

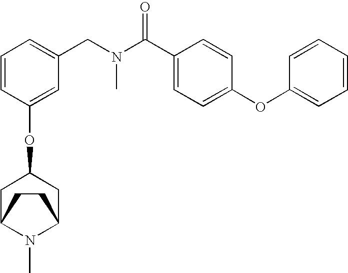

Prepared as described in Example 4 to give the title compound as a light-brown semi-solid.

¹H NMR (300.132 MHz, CDCl₃) δ 7.53-6.49 (m, 13H), 4.92-4.23 (m, 3H), 2.29 (s, 6H), 2.19-1.85 (m, 10H)

### EXAMPLE 32

### [3-((1S,3R,5R)-8-Methyl-8-aza-bicyclo[3.2.1]oct-3-yloxy)-benzyl]-(4-phenoxy-benzyl)-amine

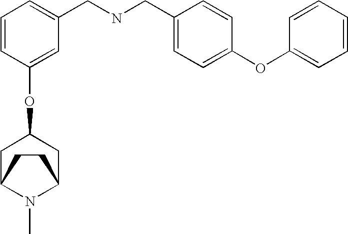

Prepared as described in example 14 to give the title compound as a light-brown semi-solid.

¹H NMR (300.132 MHz, CDCl₃) δ 7.42-6.64 (m, 13H), 4.65-4.45 (m, 1H), 3.78 (s, 4H), 2.35 (s, 3H), 2.30-1.86 (m, 10H)

### EXAMPLE 33

### N-{3-[(2,2-Dimethyl-propyl)-piperidin-4-yloxy]-benzyl}-4-phenoxy-benzamide

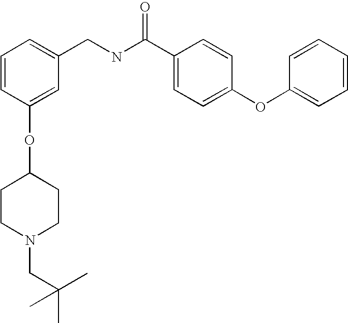

Prepared as described in Example 11 to give the title compound as a white solid.

¹H NMR (300.132 MHz, CDCl₃) δ 7.85-6.71 (m, 13H), 6.33 (s, 1H), 4.60 (d, J=5.6 Hz, 2H), 4.41-4.18 (m, 1H), 2.92-2.72 (m, 2H), 2.64-2.44 (m, 2H), 2.18 (s, 2H), 2.08-1.94 (m, 2H), 1.89-1.70 (m, 2H), 0.91 (s, 9H)

### EXAMPLE 34

### Biphenyl-4-carboxylic acid 3-((1S, 3R, 5R)-8-methyl-8-aza-bicyclo[3.2.1]oct-3-yloxy)-benzylamide

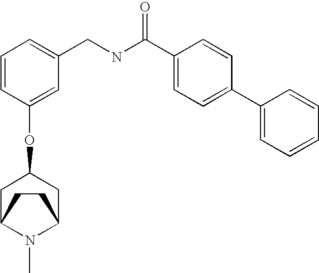

Prepared as described in Example 4 to give the title compound as a white solid.

¹H NMR (300.132 MHz, CDCl₃) δ 7.75-6.50 (m, 13H), 4.88-4.40 (m, 3H), 2.35 (s, 3H), 2.22-1.83 (m,10H)

### EXAMPLE 35

### 4′-Trifluoromethoxy-biphenyl-4-carboxylic acid 3-(1-methyl-piperidin-4-yloxy)-benzylamide

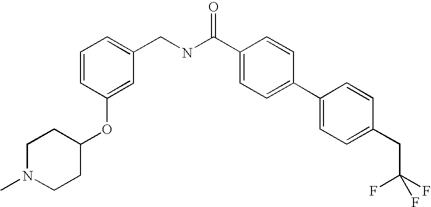

4-Bromo-N-[3-(1-methyl-piperidin-4-yloxy)-benzyl]-benzamide hydrochloride salt (0.22 gm, 0.5 mmol), 4-trifluoromethoxylphenylboronic acid (0.41 gm, 1 mmol), potassium carbonate (0.28 gm, 2 mmol) and PXPd2 (7 mg, 0.01 mmol) in 4 mL mix of 7:3:2 DME:water:ethanol was heated 150° C. for 10 min in a microwave reactor. The reaction was partitioned between methylene chloride and water. The organic phase was chromatographed on silica with a gradient elution of methanol (containing 10% NH₄OH) in methylene chloride to give the title compound as a solid.

¹H NMR (300.132 MHz, DMSO-d6) δ 9.09-9.00 (m, 1H), 8.00 (d, J=8.4 Hz, 2H), 7.86 (d, J=8.7 Hz, 2H), 7.80 (d, J=8.3 Hz, 2H), 7.48 (d, J=8.1 Hz, 2H), 7.25-7.16 (m, 1H), 6.92-6.78 (m, 3H), 4.46 (d, J=5.9 Hz, 2H), 4.36-4.26 (m, 1H), 2.64-2.53 (m, 2H), 2.18-2.08 (m, 5H), 1.95-1.84 (m, 2H), 1.68-1.52 (m, 2H)

### EXAMPLE 36

### N-[3-(1-Methyl-piperidin-4-yloxy)-benzyl]-4-thiophen-3-yl-benzamide

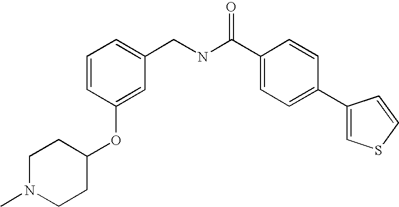

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 8.99 (t, J=5.6 Hz, 1H), 8.03-7.99 (m, 1H), 7.88 (dd, J=32.3, 8.4 Hz, 4H), 7.69-7.62 (m, 2H), 7.21 (t, J=8.0 Hz, 1H), 6.91-6.79 (m, 3H), 4.45 (d, J=5.9 Hz, 2H), 4.35-4.26 (m, 1H), 2.63-2.53 (m, 2H), 2.19-2.07 (m, 5), 1.94-1.84 (m, 2H), 1.68-1.53 (m, 2H).

### EXAMPLE 37

### 4′-Fluoro-3′-methyl-biphenyl-4-carboxylic acid 3-(1-methyl-piperidin-4-yloxy)-benzylamide

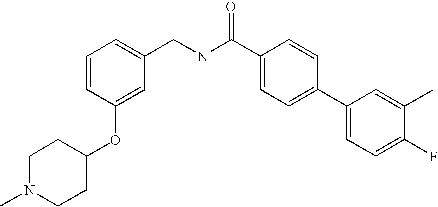

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.03 (t, J=6.0 Hz, 1H), 7.97 (d, J=7.6 Hz, 2H), 7.75 (d, J=8.4 Hz, 2H), 7.70-7.64 (m, 1H), 7.61-7.55 (m, 1H), 7.27-7.18 (m, 2H), 6.90-6.78 (m, 3H), 4.46 (d, J=5.9 Hz, 2H), 4.35-4.26 (m, 1H), 2.62-2.55 (m, 2H), 2.31 (s, 3H), 2.19-2.07 (m, 5H), 1.97-1.85 (m, 2H), 1.68-1.54 (m, 2H).

### EXAMPLE 38

### 4′-Fluoro-biphenyl-4-carboxylic acid 3-(1-methyl-piperidin-4-yloxy)-benzylamide

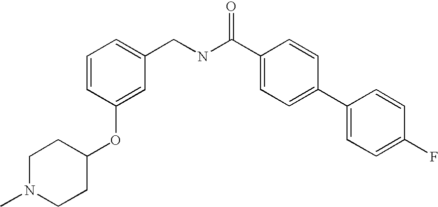

Prepared as described for 4′-Trifluoromethoxy-biphenyl-4-carboxylic acid 3-(1-methyl-piperidin-4-yloxy)-benzylamide to give the title compound as a gum.

¹H NMR (300.132 MHz, DMSO-d6) δ 9.03 (t, J=6.0 Hz, 1H), 7.98 (d, J=8.4 Hz, 2H), 7.85-7.74 (m, 3H), 7.32 (t, J=8.9 Hz, 2H), 7.22 (t, J=8.0 Hz, 2H), 6.90-6.79 (m, 3H), 4.46 (d, J=5.9 Hz, 2H), 4.36-4.25 (m, 1H), 2.64-2.54 (m, 2H), 2.19-2.07 (m, 5H), 1.96-1.84 (m, 2H), 1.68-1.54 (m, 2H)

### EXAMPLE 39

### 4′-Chloro-biphenyl-4-carboxylic acid 3-(1-methyl-piperidin-4-yloxy)-benzylamide

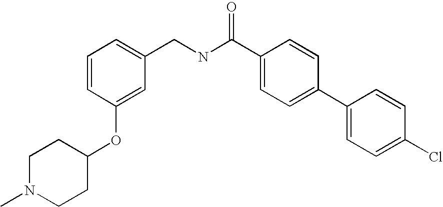

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.04 (t, J=5.9 Hz, 1H), 7.99 (d, J=8.5 Hz, 2H), 7.82-7.74 (m, 4H), 7.55 (d, J=8.6 Hz, 2H), 7.22 (t, J=8.0 Hz, 1H), 6.91-6.79 (m, 3H), 4.46 (d, J=5.9 Hz, 2H), 4.37-4.26 (m, 1H), 2.63-2.54 (m, 2H), 2.19-2.08 (m, 5H), 1.96-1.84 (m, 2H), 1.68-1.55 (m, 2H)

### EXAMPLE 40

### 4′-Methoxy-biphenyl-4-carboxylic acid 3-(1-methyl-piperidin-4-yloxy)-benzylamide

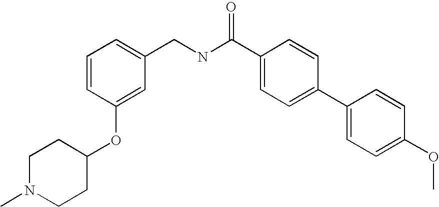

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 8.99 (t, J=6.0 Hz, 1H), 7.95 (d, J=8.5 Hz, 2H), 7.76-7.66 (m, 4H), 7.22 (t, J=8.0 Hz, 1H), 7.05 (d, J=8.8 Hz, 2H), 6.91-6.79 (m, 3H), 4.46 (d, J=6.0 Hz, 2H), 4.36-4.26 (m, 1H), 3.81 (s, 3H), 2.63-2.54 (m, 2H), 2.19-2.07 (m, 5H), 1.97-1.84 (m, 2H), 1.68-1.55 (m, 2H)

### EXAMPLE 41

### 4′-Methyl-biphenyl-4-carboxylic acid 3-(1-methyl-piperidin-4-yloxy)-benzylamide

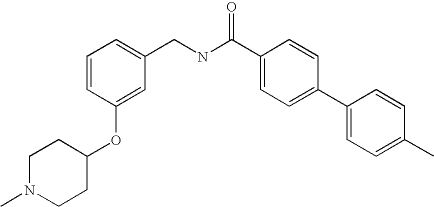

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.01 (t, J=6.0 Hz, 1H), 7.97 (d, J=8.5 Hz, 2H), 7.75 (d, J=8.4 Hz, 2H), 7.63 (d, J=8.2 Hz, 2H), 7.30 (d, J=8.0 Hz, 2H), 7.22 (t, J=8.0 Hz, 2H), 6.91-6.78 (m, 3H), 4.46 (d, J=5.9 Hz, 2H), 4.36-4.26 (m, 1H), 2.64-2.54 (m, 2H), 2.35 (s, 3H), 2.18-2.08 (m, 5H), 1.96-1.85 (m, 2H), 1.67-1.54 (m, 2H).

### EXAMPLE 42

### 3′-Chloro-biphenyl-4-carboxylic acid 3-(1-methyl-piperidin-4-yloxy)-benzylamide

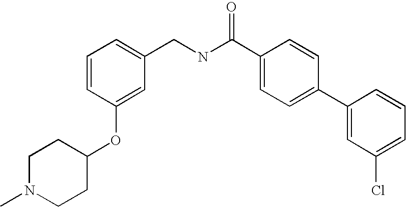

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.06 (t, J=5.9 Hz, 1H), 8.00 (d, J=8.4 Hz, 2H), 7.85-7.78 (m, 3H), 7.74-7.69 (m, 1H), 7.56-7.44 (m, 2H), 7.22 (t, J=8.0 Hz, 1H), 6.91-6.79 (m, 3H), 4.46 (d, J=5.9 Hz, 2H), 4.38-4.27 (m, 1H), 2.64-2.55 (m, 2H), 2.21-2.08 (m, 5H), 1.98-1.84 (m, 2H), 1.70-1.56 (m, 2H)

### EXAMPLE 43

### 4′-Methanesulfonyl-biphenyl-4-carboxylic acid 3-(1-methyl-piperidin-4-yloxy)-benzylamide

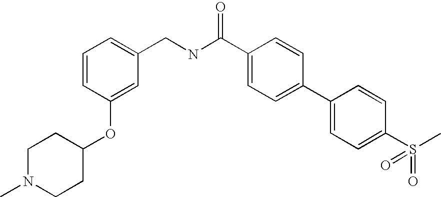

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.09 (t, J=6.0 Hz, 1H), 8.07-7.97 (m, 5H), 7.88 (d, J=8.4 Hz, 2H), 7.22 (t, J=8.0 Hz, 1H), 6.92-6.79 (m, 3H), 4.47 (d, J=5.9 Hz, 2H), 4.37-4.26 (m, 1H), 3.26 (s, 3H), 2.63-2.54 (m, 2H), 2.19-2.08 (m, 5H), 1.95-1.85 (m, 2H), 1.69-1.54 (m, 2H)

### EXAMPLE 44

### N-[3-((1S,3R,5R)-8-Methyl-8-aza-bicyclo[3.2.1]oct-3-yloxy)-benzyl]-4-pyridin-4-yl-benzamide

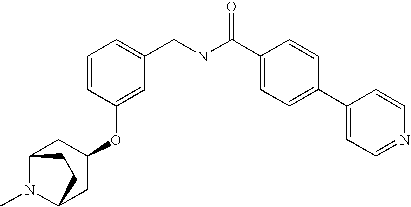

Prepared as described for 4′-Trifluoromethoxy-biphenyl-4-carboxylic acid 3-(1-methyl-piperidin-4-yloxy)-benzylamide to give the title compound as a solid.

¹H NMR (300.132 MHz, DMSO-d6) δ 9.09 (t, J=5.9 Hz, 1H), 8.67 (dd, J=4.5, 1.6 Hz, 2H), 7.98 (dd, J=32.3, 8.5 Hz, 4H), 7.84-7.66 (m, 2H), 7.22 (t, J=7.9 Hz, 1H), 6.90-6.78 (m, 2H), 6.75-6.70 (m, 1H), 4.55-4.49 (m, 2H), 4.48-4.41 (m, 24H), 3.04-2.98 (m, 2H), 2.17 (s, 2H), 2.05-1.87 (m, 5H), 1.79-1.69 (m, 2H)

### EXAMPLE 45

### 4′-Fluoro-biphenyl-4-carboxylic acid 3-((1S,3R,5R)-8-methyl-8-aza-bicyclo[3.2.1]oct-3-yloxy)-benzylamide

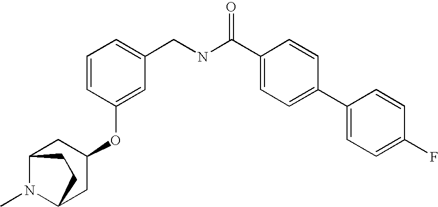

Prepared as described for example 4 to give the title compound as a solid.

¹H NMR (300.132 MHz, CDCl₃) δ 7.85 (d, J=8.4 Hz, 2H), 7.63-7.53 (m, 4H), 7.31-7.22 (m, 2H), 7.14 (t, J=8.7 Hz, 2H), 6.93 (d, J=7.6 Hz, 1H), 6.85-6.82 (m, 1H), 6.78-6.74 (m, 1H), 6.41-6.34 (m, 1H), 4.63 (d, J=5.6 Hz, 2H), 4.53 (t, J=4.9 Hz, 1H), 3.14 (s, 2H), 2.32 (s, 3H), 2.22-1.89 (m, 8H)

### EXAMPLE 46

### 4′-Dimethylamino-biphenyl-3-carboxylic acid 3-(1-methyl-piperidin-4-yloxy)-benzylamide

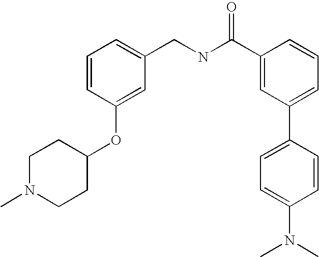

Prepared as described for 4′-Trifluoromethoxy-biphenyl-4-carboxylic acid 3-(1-methyl-piperidin-4-yloxy)-benzylamide using 3-Bromo-N-[3-(1-methyl-piperidin-4-yloxy)-benzyl]-benzamide hydrochloride salt to give the title compound as a gum.

¹H NMR (400.131 MHz, DMSO-d6) δ 9.06 (t, J=5.9 Hz, 1H), 8.09 (s, 1H), 7.74 (d, J=9.1 Hz, 2H), 7.59 (d, J=8.8 Hz, 2H), 7.49 (t, J=7.7 Hz, 1H), 7.22 (t, J=7.8 Hz, 1H), 6.92-6.78 (m, 5H), 4.47 (d, J=5.9 Hz, 2H), 4.34-4.26 (m, 1H), 2.95 (s, 6H), 2.61-2.54 (m, 2H), 2.17-2.06 (m, 5H), 1.94-1.85 (m, 2H), 1.65-1.55 (m, 2H)

### EXAMPLE 47

### N-Methyl-N-[3-(1-methyl-piperidin-4-yloxy)-benzyl]-4-phenoxy-benzamide

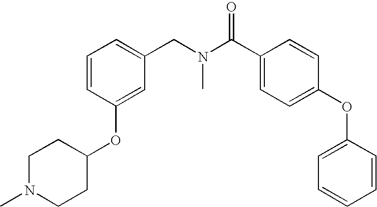

Prepared as described in example 8 to give the title compound as an oil.

¹H NMR (300.132 MHz, DMSO-d6) δ 7.50-7.38 (m, 4H), 7.29-7.16 (m, 3H), 7.11-6.98 (m, 4H), 6.89-6.74 (m, 2H), 4.63-4.48 (m, 2H), 4.38-4.27 (m, 1H), 2.88 (s, 3H), 2.66-2.56 (m, 2H), 2.21-2.10 (m, 5H), 1.98-1.84 (m, 2H), 1.70-1.54 (m, 2H)

### EXAMPLE 48

### N-[3-(1-Cyclopropyl-piperidin-4-yloxy)-benzyl]-4-phenoxy-benzamide

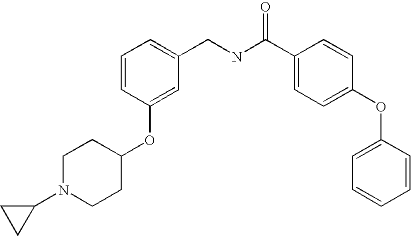

Prepared as described in Example 11 using [(1-ethoxycyclopropyl)oxy]trimethylsilane as the carbonyl equivalent to give the title compound as a solid.

¹H NMR (300.132 MHz, DMSO-d6) δ 8.91 (t, J=5.9 Hz, 1H), 7.92 (d, J=8.8 Hz, 2H), 7.44 (t, J=8.0 Hz, 2H), 7.21 (t, J=7.6 Hz, 2H), 7.06 (t, J=10.6 Hz, 4H), 6.90-6.78 (m, 3H), 4.43 (d, J=5.9 Hz, 2H), 4.37-4.28 (m, 1H), 2.83-2.74 (m, 2H), 2.44-2.33 (m, 2H), 1.92-1.81 (m, 2H), 1.66-1.47 (m, 3H), 0.44-0.37 (m, 2H), 0.31-0.24 (m, 2H)

### EXAMPLE 49

### 4-Phenoxy-N-{3-[1-(tetrahydro-furan-3-yl)-piperidin-4-yloxy]-benzyl}-benzamide

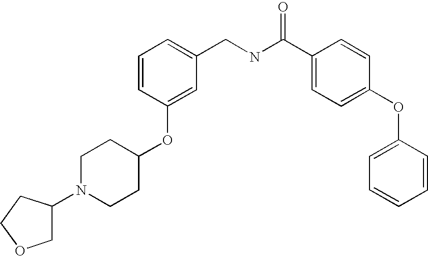

Prepared as described in Example 11 to give the title compound as a solid.

¹H NMR (300.132 MHz, DMSO-d6) δ 8.95-8.87 (m, 1H), 7.92 (d, J=8.6 Hz, 2H), 7.44 (t, J=7.9 Hz, 2H), 7.21 (t, J=7.8 Hz, 2H), 7.12-7.02 (m, 4H), 6.90-6.78 (m, 3H), 4.43 (d, J=5.7 Hz, 2H), 4.35-4.27 (m, 1H), 3.82-3.71 (m, 2H), 3.68-3.58 (m, 1H), 3.48-3.41 (m, 1H), 2.96-2.83 (m, 1H), 2.76-2.66 (m, 1H), 2.62-2.51 (m, 2H), 2.30-2.11 (m, 2H), 2.00-1.85 (m, 2H), 1.78-1.50 (m, 3H)

Although the foregoing invention has been described in some detail by way of illustration and example for purposes of clarity of understanding, it will be readily apparent to those of ordinary skill in the art in light of the teachings of this invention that changes and modifications may be made thereto without departing from the spirit or scope of the disclosure.

1. A compound in accord with Formula I: 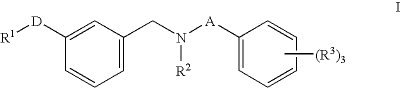
wherein:
D is selected from —CH₂— or —O—, and
R¹ is selected from -C₁₋₆alkylene-NR⁵R⁶ wherein R⁵ and R⁶ are independently at each occurrence selected from hydrogen or -C₁₋₆alkyl, or R⁵ and R⁶ together with the N to which they are attached are selected from morpholino or a moiety of Formula II 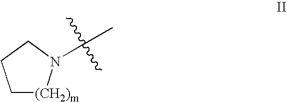
where m is 1, 2 or 3, and the moiety of Formula II may be substituted with ═O;
or, R¹ is selected from: 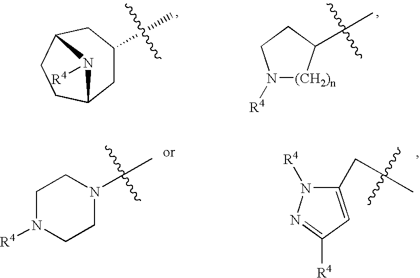
wherein R⁴ is selected from hydrogen, -C₁₋₆alkyl, -C₃₋₈cycloalkyl, -C₃₋₈cyclooxyalkyl or benzyl and n is 1, 2 or 3,
R² is selected from hydrogen, -C₁₋₆alkyl or C₃₋₈cycloalkyl;
A is selected from —CH₂— or —C(═O)—;
R³ is selected independently at each occurrence from hydrogen, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷, —CO₂R⁷, -C₁₋₆alkyl, -C₂₋₆alkenyl, -C₂₋₆alkynyl, -C₁₋₆alkoxy, -C₃₋₈cycloalkyl, —O—CH₂—O—, or —G—Ar,
wherein G is —O—, —CH₂—, —O—CH₂— or a bond, and
Ar is selected from a 5- or 6-membered aromatic or heteroaromatic ring having 0, 1 or 2 nitrogen atoms, 0 or 1 oxygen atoms, and 0 or 1 sulfur atoms, or is selected from an 8-, 9- or 10-membered fused aromatic or heteroaromatic ring system having 0, 1, 2 or 3 nitrogen atoms, 0 or 1 oxygen atoms, and 0 or 1 sulfur atoms;
wherein Ar is unsubstituted or has 1, 2 or 3 substituents independently selected at each occurrence from -C₁₋₆alkyl, -C₂₋₆alkenyl, -C₂₋₆alkynyl, halogen, —CN, —NO₂, —CF₃, —CONR⁷R⁸, —S(O)ₙR⁷, —NR⁷R⁸, —CH₂NR⁷R⁸, —OR⁷, —CH₂OR⁷, —NC(═O)R⁷ or —CO₂R⁷;
wherein R⁷ and R⁸ are independently selected from hydrogen, -C₁₋₆alkyl, -C₁₋₆alkoxy or -C₃₋₈cycloalkyl,
or an in vivo-hydrolysable precursor or pharmaceutically-acceptable salt thereof, with the proviso that said compound is not N-[3-(1-methyl-piperidin-4-yloxy)-benzyl]-3-phenoxy-benzamide or N-[3-(1-methyl-piperidin-4-yloxy)-benzyl]-4-phenoxy-benzamide.

2. A compound according to claim 1, wherein:
D is —O—.

3. A compound according to claim 1, wherein:
A is —C(═O)—.

4. A compound according to claim 1, wherein:
D is selected from —CH₂— or —O—, and
R¹ is selected from:

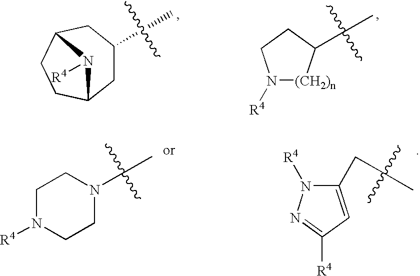

5. A compound selected from:
N-[3-((1R,3R,5S)-8-Methyl-8-aza-bicyclo[3.2.1]oct-3-yloxy)-benzyl]-4-phenoxy-benzamide hydrochloride;
N-[3-((1R,3R,5S)-8-Methyl-8-aza-bicyclo[3.2.1]oct-3-yloxy)-benzyl]-4-propyl-benzamide;
4-Cyclohexyl-N-[3-((1R,3R,5S)-8-methyl-8-aza-bicyclo[3.2.1]oct-3-yloxy)-benzyl]-benzamide;
4-Benzyl-N-[3-(1-methyl-piperidin-4-yloxy)-benzyl]-benzamide;
4-Benzyloxy-N-[3-(1-methyl-piperidin-4-yloxy)-benzyl]-benzamide;
Biphenyl-4-carboxylic acid 3-(1-methyl-piperidin-4-yloxy)-benzylamide;
N-[3-(1-Benzyl-piperidin-4-yloxy)-benzyl]-4-phenoxy-benzamide;
4-Phenoxy-N-[3-(piperidin-4-yloxy)-benzyl]-benzamide;
4-Phenoxy-N-[3-(1-ethyl-piperidin-4-yloxy)-benzyl]-benzamide;
4-Phenoxy-N-[3-(1-propyl-piperidin-4-yloxy)-benzyl]-benzamide;
N-Ethyl-N-[3-(1-ethyl-piperidin-4-yloxy)-benzyl]-4-phenoxy-benzamide;
[3-(1-Methyl-piperidin-4-yloxy)-benzyl]-(4-phenoxy-benzyl)-amine;
(4-Isopropyl-benzyl)-[3-(1-methyl-piperidin-4-yloxy)-benzyl]-amine;
Benzo[1,3]dioxol-5-ylmethyl-[3-(1-methyl-piperidin-4-yloxy)-benzyl]-amine;
(4-Chloro-benzyl)-[3-(1-methyl-piperidin-4-yloxy)-benzyl]-amine;
Methyl-[3-(1-methyl-piperidin-4-yloxy)-benzyl]-(4-phenoxy-benzyl)-amine;
N-[3-(4-Methyl-piperazin-1-ylmethyl)-benzyl]-4-phenoxy-benzamide;
4′-Methoxy-biphenyl-4-carboxylic acid methyl-[3-(1-methyl-piperidin-4-yloxy)-benzyl]-amide;
4′-Methoxy-biphenyl-4-carboxylic acid methyl-[3-((1S,3R,5R)-8-methyl-8-aza-bicyclo[3.2.1]oct-3-yloxy)-benzyl]-amide;
4′-Methoxy-biphenyl-4-carboxylic acid 3-((1S,3R,5R)-8-methyl-8-aza-bicyclo[3.2.1]oct-3-yloxy)-benzylamide;
4-Cyclohexyl-N-methyl-N-[3-(1-methyl-piperidin-4-yloxy)-benzyl]-benzamide;
Biphenyl-4-ylmethyl-[3-(1-methyl-piperidin-4-yloxy)-benzyl]-amine;
Biphenyl-4-carboxylic acid methyl-[3-(1-methyl-piperidin-4-yloxy)-benzyl]-amide;
[3-((1S,3R,5R)-8-Methyl-8-aza-bicyclo[3.2.1]oct-3-yloxy)-benzyl]-(3-phenoxy-benzyl)-amine;
Methyl-[3-((1S,3R,5R)-8-methyl-aza-bicyclo[3.2.1]oct-3-yloxy)-benzyl]-(4-phenoxy-benzyl)-amine;
Biphenyl-4-ylmethyl-[3-((1S,3R,5R)-8-methyl-8-aza-bicylc0[3.2.1]oct-3-yloxy)-benzyl]-amine;
N-Methyl-N-[((1S,3R,5R)-8-methyl-8-aza-bicycl0[3.2.1]oct-3-yloxy)-benzyl]-3-phenoxy-benzamide;
Biphenyl-4-carboxylic acid 3-((1S,3R,5R)-8-methyl-8-aza-bicyclo[3.2.1]oct-3-yloxy)-benzamide;
N-Methyl-N-[((1S,3R,5R)-8-methyl-8-aza-bicycl0[3.2.1]oct-3-yloxy)-benzyl]-4-phenoxy-benzamide;
[3-((1S,3R,5R)-8-Methyl-8-aza-bicyclo[3.2.1]oct-3-yloxy)-benzyl]-(4-phenoxy-benzyl)-amine;
N-{3-[(2,2-Dimethyl-propyl)-piperidin-4-yloxy]-benzyl}-4-phenoxy-benzamide;
Biphenyl-4-carboxylic acid 3-((1S, 3R, 5R)-8-methyl-8-aza-bicyclo[3.2.1]oct-3-yloxy)-benzylamide;
4′-Trifluoromethoxy-biphenyl-4-carboxylic acid 3-(1-methyl-piperidin-4-yloxy)-benzylamide;
N-[3-(1-Methyl-piperidin-4-yloxy)-benzyl]-4-thiophen-3-yl-benzamide;
4′-Fluoro-3′-methyl-biphenyl-4-carboxylic acid 3-(1-methyl-piperidin-4-yloxy)-benzylamide;
4′-Fluoro-biphenyl-4-carboxylic acid 3-(1-methyl-piperidin-4-yloxy)-benzylamide;
4′-Chloro-biphenyl-4-carboxylic acid 3-(1-methyl-piperidin-4-yloxy)-benzylamide;
4′-Methoxy-biphenyl-4-carboxylic acid 3-(1-methyl-piperidin-4-yloxy)-benzylamide;
4′-Methyl-biphenyl-4-carboxylic acid 3-(1-methyl-piperidin-4-yloxy)-benzylamide;
3′-Chloro-biphenyl-4-carboxylic acid 3-(1-methyl-piperidin-4-yloxy)-benzylamide;
4′-Methanesulfonyl-biphenyl-4-carboxylic acid 3-(1-methyl-piperidin-4-yloxy)-benzylamide;
N-[3-((1S,3R,5R)-8-Methyl-8-aza-bicyclo[3.2.1]oct-3-yloxy)-benzyl]-4-pyridin-4-yl-benzamide;
4′-Fluoro-biphenyl-4-carboxylic acid 3-((1S,3R,5R)-8-methyl-8-aza-bicyclo[3.2.1]oct-3-yloxy)-benzylamide;
4′-Dimethylamino-biphenyl-3-carboxylic acid 3-(1-methyl-piperidin-4-yloxy)-benzylamide;
N-Methyl-N-[3-(1-methyl-piperidin-4-yloxy)-benzyl]-4-phenoxy-benzamide;
N-[3-(1-Cyclopropyl-piperidin-4-yloxy)-benzyl]-4-phenoxy-benzamide, and
4-Phenoxy-N-{3-[1-(tetrahydro-furan-3-yl)-piperidin-4-yloxy]-benzyl}-benzamide;
or an in vivo-hydrolysable precursor or pharmaceutically-acceptable salt thereof.

6. A method of treatment or prophylaxis of a disease or condition in which modulation of the MCH1 receptor is beneficial which method comprises administering to a subject suffering from said disease or condition a therapeutically-effective amount of a compound according to claim 1.

7. The method of claim 6, wherein said disease or condition is selected from mood changes, anxiety, depression, generalized anxiety disorder, panic attacks, panic disorder, obsessive-compulsive disorder and bipolar disorders, obesity and related disorders, eating disorders, psychiatric disorders, neurological disorders, and pain.

8. (canceled)

9. A pharmaceutical composition comprising a pharmaceutically-acceptable diluent, lubricant or carrier and a compound according to claim 1.

10. A method of treatment or prophylaxis of a disease or condition in which modulation of the MCH1 receptor is beneficial which method comprises administering a therapeutically-effective amount of a pharmaceutical composition according to claim 9 to a subject suffering from said disease or condition.

11. The method of claim 10, wherein said disease or condition is selected from mood changes, anxiety, depression, generalized anxiety disorder, panic attacks, panic disorder, obsessive-compulsive disorder and bipolar disorders, obesity and related disorders, eating disorders, psychiatric disorders, neurological disorders, and pain.

12-15. (canceled)

16. A method of treatment or prophylaxis of a disease or condition in which modulation of the MCH1 receptor is beneficial which method comprises administering to a subject suffering from said disease or condition a therapeutically-effective amount of a compound according to claim 5.

17. The method of claim 16, wherein said disease or condition is selected from mood chances, anxiety, depression, generalized anxiety disorder, panic attacks, panic disorder, obsessive-compulsive disorder and bipolar disorders, obesity and related disorders, eating disorders, psychiatric disorders, neurological disorders, and pain.

18. A pharmaceutical composition comprising a pharmaceutically-acceptable diluent, lubricant or carrier and a compound according to claim 5.

19. A compound according to claim 1, wherein R² is H or -C₁₋₆alkyl.

20. A compound according to claim 1, wherein R¹ is 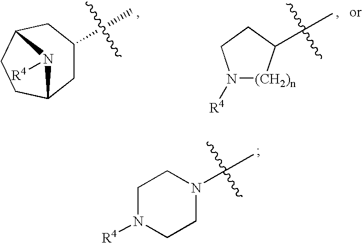
n is 2; and R⁴ is -C₁₋₆alkyl.

21. A compound according to claim 20, wherein R¹ is

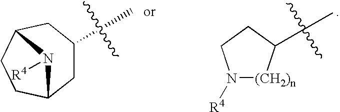

22. A compound according to claim 20, wherein R⁴ is methyl, ethyl, or propyl, 2,2-dimethyl-propyl.

23. A compound according to claim 1, wherein R⁴ is hydrogen, methyl, ethyl, propyl, 2,2-dimethyl-propyl, benzyl, cyclopropyl, or tetrahydro-furan-3-yl.

24. A compound according to claim 1, wherein R¹ is 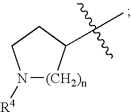
R⁴ is hydrogen, methyl, ethyl, propyl, 2,2-dimethyl-propyl, benzyl, cyclopropyl, or tetrahydro-furan-3-yl; and n is 2.

25. A compound according to claim 1, wherein R¹ is 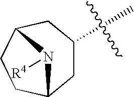
and R⁴ is methyl.

26. A compound according to claim 1, wherein R¹ is 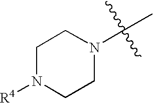
and R⁴ is methyl.

27. A compound according to claim 1, wherein R³ is selected independently at each occurrence from hydrogen, halogen, -C₁₋₆alkyl, -C₃₋₈cycloalkyl, or —G—Ar.

28. A compound according to claim 27, wherein G is a bond or O.

29. A compound according to claim 28, wherein Ar is a 6-membered aromatic, wherein said Ar is unsubstituted or has 1 substituent independently selected from -C₁₋₆alkyl, halogen, —S(O)ₙR⁷, —NR⁷R⁸, or —R⁷; R⁷ and R⁸ are each independently -C₁₋₆alkyl; and n is 2.

30. A compound according to claim 1, wherein R³ is —G—Ar; G is a bond or O; and Ar is a 6-membered aromatic, wherein said Ar is unsubstituted or has 1 substituent independently selected from -C₁₋₆alkyl, halogen, —S(O)ₙR⁷, —NR⁷R⁸, or —OR⁷, wherein R⁷ and R⁸ are each independently -C₁₋₆alkyl and n is 2.
